(19) United States
(12) Patent Application Publication
(10) Pub. No.: US 2012/0034214 A1
(43) Pub. Date: Feb. 9, 2012

(54) METHODS AND KITS FOR DETERMINING THE PROGNOSIS OF PULMONARY SARCOIDOSIS

(75) Inventor: Ling-Pei HO, Oxford (GB)

(73) Assignee: ISIS Innovation Limited, Summertown (GB)

(21) Appl. No.: 13/189,007

(22) Filed: Jul. 22, 2011

Related U.S. Application Data
(60) Provisional application No. 61/366,872, filed on Jul. 22, 2010.

(51) Int. Cl. A61K 39/395; C40B 30/04; A61K 45/00; C07H 21/00; A61P 37/06; A61K 31/56; A61P 35/00; C12Q 1/68

(52) U.S. Cl. 424/133.1; 506/9; 435/6.12; 536/24.31; 514/169; 424/184.1

(57) ABSTRACT

The invention provides a method for determining the prognosis of pulmonary sarcoidosis in an individual subject, comprising conducting gene expression analysis on a sample from the subject. The sample is obtained by bronchoscopy under procedural methods not requiring general anaesthesia.

### CROSS-REFERENCE TO RELATED APPLICATIONS

This application claims priority benefit of U.S. Provisional Patent Application 61/366,872, filed Jul. 22, 2010, which is hereby incorporated herein by reference in its entirety.

### FIELD OF THE INVENTION

The invention relates to determining the prognosis of pulmonary sarcoidosis.

### BACKGROUND OF THE INVENTION

Sarcoidosis is a multisystem granulomatous inflammatory disease of unknown aetiology, characterized by non-caseating granulomatous deposits and intense T cell activity. Almost any organs can be affected but the lungs are by far the most common. Approximately 95% of patients have computed tomographic evidence of deposits in the lungs. Sarcoidosis affecting the lungs is known as pulmonary sarcoidosis.

Significant advances have been made to characterize the immunological features of sarcoidosis, but the central immunological mechanisms remain unclear. There is evidence pointing to overactivity of TH1-biased CD4 T cells as the initial abnormality leading to activation of macrophages and downstream formation of granuloma. It is also possible that the macrophage is the primary cellular abnormality, and an inability to degrade an intracellular antigen causes the formation and persistence of granuloma. Overall, there is general agreement that the condition is caused by a complex genetic susceptibility to granulomagenesis, triggered by an unknown inhaled antigen. Genome wide association studies and directed SNP searches have identified ANXA11 (annexin A11) and BTNL-2 as susceptibility genes, both of which are related to control of T cell activity.

The symptoms of sarcoidosis usually appear gradually but can occasionally appear suddenly. The clinical course varies dramatically. Most patients with pulmonary sarcoidosis have a good prognosis. The immune disturbances and granulomatous deposits resolve spontaneously over 2 to 5 years. This is known as self-limiting sarcoidosis. However about 30% of patients have progressive disease leading to varying degrees of pulmonary fibrosis and even death in 1 to 5% of cases. This is know as progressive sarcoidosis. Currently almost nothing is known of the cause of this progression in disease and with the exception of patients who present with Loefgren's syndrome, there are no markers to differentiate between these two groups of patients. Indeed, both groups of patients often present with very similar (or no) symptoms.

There is a long-standing need for a reliable means of separating patients with self-limiting from those with progressive pulmonary sarcoidosis. That is, there is a need for a means for determining which patients will have progressive pulmonary sarcoidosis.

### SUMMARY OF THE INVENTION

The invention provides a method for determining the prognosis of pulmonary sarcoidosis in an individual subject, comprising conducting gene expression analysis on a sample from the subject, wherein said subject has or is suspected of having pulmonary sarcoidosis; and said sample is obtained by bronchoscopy under procedural methods not requiring general anesthesia, preferably a transbronchial lung biopsy (TBB). Gene expression analysis typically comprises determining the expression level of one or more genes.

In one embodiment, the method of the invention comprises categorising the sarcoidosis as either progressive or self-limiting. Typically, this embodiment is carried out by a method comprising:
(a) measuring the expression level of at least one gene set in the sample taken from the subject;
(b) comparing the measured expression level to the expression level for the same at least one gene set as previously determined in:
   patients confirmed as having progressive sarcoidosis, and/or
   patients confirmed as having self-limiting sarcoidosis and
(c) thereby categorising the sarcoidosis in said subject as either progressive or self-limiting.

In one embodiment, the invention provides a method for determining the prognosis of pulmonary sarcoidosis in an individual subject, comprising conducting gene expression analysis on a sample from the subject,

wherein said subject has or is suspected of having pulmonary sarcoidosis and wherein said gene expression analysis comprises determining the expression level of:
the gene set consisting of the genes listed in Table 1 or Table 3; or
at least one gene set from those listed in Table 2 or Table 5; or
at least one gene set selected from the “immune response”, “immune system process”, “lymphocyte activation”, “defence response”, and “leukocyte activation” gene sets, or any combination of said gene sets; or
at least one gene set from each of the major categories shown in Table 5.

In one embodiment, the invention provides a method of slowing or preventing the development of pulmonary fibrosis in an individual subject, comprising;

(i) assessing the prognosis of pulmonary sarcoidosis in the subject using a method according to the invention; and
(ii) administering to a subject having sarcoidosis categorised in (i) as progressive, a therapeutically effective amount of an agent suitable for the treatment of pulmonary fibrosis.

In one embodiment, the invention provides a test kit for use in a method for determining the prognosis of pulmonary sarcoidosis in an individual subject, which test kit comprises one or more agents suitable for analysing gene expression in a sample taken from the subject.

### DESCRIPTION OF THE FIGURES

FIG. 1. Gene expression profile for progressive, fibrotic (P-F) and self-limiting (N-SL) patients. Panel A. Expression profile for the two groups of patients showing 334 differentially expressed genes (p<0.01, Bayesian moderated t-test). Red areas indicate increased gene expression relative to mean of all samples, and green decreased expression. Each column represents one patient, and each row a gene. In general, all genes above the horizontal line are relatively over-expressed in N-SL patients and relatively under-expressed in P-F patients. Genes below the horizontal line are relatively over-expressed in P-F patients and relatively under-expressed in N-SL patients. Hierarchical clustering was used to show the degree of similarity of overall gene expression profile between each patient (top dendogram) and the left dendogram shows the degree of similarity between the genes. Panel B. Top 50 genes with increased expression in P-F compared to N-SL group, ranked by fold change (top panel), and all 51 down regulated genes in P-F compared to N-SL by fold change (Panel C); p values from Bayesian moderated t test. The full list of differentially expressed genes is given in Table 3.

FIG. 2. GSEA analysis of microarray gene signals between N-SL and P-F groups, using Gene Ontology (GO) defined gene-sets. Panel A shows the top 5 GO-defined gene-sets that were significantly enriched among genes up-regulated in the P-F group (FDR q<0.001). The red-blue strip represents the 26,626 genes expressed in the samples ranked according to differential expression between the N-SL and P-F groups (hence phenotype). Increasing gene signal in the N-SL is towards the left end of the bar, increasing gene signal in the P-F group is towards the right. Each line in the ‘bar code’ represents presence of a gene from the GO-defined gene-set (eg immune system process gene set) within the ranked genes. The enrichment score (y axis) is a running-sum statistic which increases when the gene in that particular gene-set is found in the ranked gene list and decreases otherwise. The changes are weighted such that the magnitude is greater if there is a strong correlation of the gene from the defined gene-set with phenotype (i.e. found towards the extreme ends of the ranked list) The leading edge subset is defined as genes appearing at or before the running sum reaches maximum deviation from zero. Panel B. Mean intensity of expression of leading edge genes from gene-sets with FDR q<0.01 for each patient. Each row represents a gene-set and shows the mean expression of leading genes from that gene-set, and each column is a patient, clustered by hierachical method to reflect degree of similarity. The gene-sets are grouped according to biological categories, and the panel shows the 10 main categories for these gene-sets (composition of these categories can be found in Table 5). The bar at the top indicates the relative expression in the N-SL versus P-F groups, with negative scores (relative under-expression) being shown for the N-SL group to the left, and positive scores (relative over-expression) being shown for the P-F group to the right.

FIG. 3. Quality of lung biopsy samples using a routine clinical tool (bronchoscopic transbronchial biopsy). Panel A shows RNA quality (18S and 28S profiles), showing minimal degradation of RNA from tissue sample, and comparative quality for all fifteen patients. Panel B. Comparison of mean gene expression signal for all genes in both groups. Panel C. Boxplot for RMA normalised intensity for all the genes for each patient. “1_Good” refers to a N-SL patient in Set 1; “2_Poor” refers a P-F patient from Set 2. Panel D. Fold change of 5 random genes, comparing mean expression for each gene in N-SL to P-F group, using qPCR and DNA microarray to determine gene expression.

### DESCRIPTION OF THE TABLES

Table 1. A list of the top 50 individual genes found to have increased expression in progressive-fibrotic sarcoidosis (PF) patients compared to nodular, self-limiting sarcoidosis (N-SL) patients, ranked by fold change (FC) (top panel); and all 52 down-regulated genes in PF compared to N-SL patients, ranked by fold change (bottom panel). p values are from a Bayesian moderated t test.

Table 2. A list of the 43 gene sets that were most significantly enriched in terms of up-regulated expression of their component genes in P-F patients compared to N-SL patients (FDR q value q<0.0001).

Table 3. Full list of genes found differentially expressed between the two groups of patients with p<0.01 (Bayesian moderated t-test). FC refers to fold change as of the average normalised-intensity for that gene in N-SL group compared to P-F group.

Table 4. Full list of gene ontology-defined biological categories found in P-F patients with their normalised enrichment score (NES) and FDR q values.

Table 5. A list of the gene sets that were most significantly enriched in terms of up-regulated expression of their component genes in P-F patients compared to N-SL patients (FDR q value q<0.01), grouped in major categories on the basis of overlapping leading edge genes, and being directly related terms in the gene ontology hierarchy.

Table 6. Complete list of the genes in the gene sets of Tables 2, 4 and 5.

### DETAILED DESCRIPTION OF THE INVENTION

### Subjects

The present invention relates to a method for determining the prognosis of pulmonary sarcoidosis in an individual subject. More specifically, the invention relates to determining whether the individual has progressive or self-limiting pulmonary sarcoidosis. The individual under test typically has or is suspected of having pulmonary sarcoidosis. The individual is typically a mammal. The mammal is typically a human or a domestic mammal such as a horse, a cow, a sheep, a dog or a cat. The individual is preferably a human.

The individual may have been diagnosed as having pulmonary sarcoidosis, or be suspected of having pulmonary sarcoidosis, because they display one or more symptoms commonly associated with sarcoidosis. However, the subject may display no such symptoms. The common symptoms of sarcoidosis are vague, and can sometimes be similar to symptoms of lymphoma and tuberculosis. Typically, a sarcoidosis patient will present with clinical features in accordance to those set out in the Statement on Sarcoidosis published by the American Thoracic Society (Am J Respir Crit Care Med. 1999; 160(2):736-55).

Typically, a sarcoidosis patient will present with one or more of the following symptoms:
Symptoms associated with the lung: selected from shortness of breath, wheezing, hoarseness, dry or productive cough, chest pain, and tightness in the chest;
Symptoms associated with the eye: selected from uveitis, uveoparotitis, iridocyclitis, retinal inflammation, loss of visual acuity or blindness, eye dryness, redness, tearing, burning or itching, and photophobia;
Cutaneous symptoms: selected from range from rashes, nodules, erythema nodosum and lupus pernio;
General symptoms: selected from joint and muscle pain, irregular heartbeat, loss of sensation, loss of muscle strength, headache, dizziness, weight loss, fever and fatigue.

Patients with suspected sarcoidosis are typically assessed with a chest radiograph for pulmonary involvement. The radiograph may be generated by, for example, X-ray, CT, MRI or PET scan, typically CT scan. Radiographs are typically assigned a stage of 0-4 according to the presence or absence of hilar adenopathy and parenchymal disease, e.g., pulmonary infiltration or fibrosis. Thus there are five stages:
Stage 0: no visible intrathoracic findings;
Stage 1: bilateral hilar lymphadenopathy (BHL), which may be accompanied by paratracheal adenopathy. Lung fields are clear of infiltrates.
Stage 2: bilateral hilar adenopathy (BHL) accompanied by parenchymal infiltration;
Stage 3: parenchymal infiltration without bilateral hilar adenopathy (BHL)
Stage 4: advanced pulmonary fibrosis with evidence of honey-combing, hilar retraction, bullae, cysts, and emphysema.

Subjects tested using the present invention will typically produce radiographs which are assigned to stages 1 to 4.

Typically, an individual subject may be suspected of having pulmonary sarcoidosis because of the presence of one or more symptoms associated with the lung (typically cough or breathlessness) and/or abnormalities on a chest radiograph.

### Prognosis

The present invention involves conducting gene expression analysis on a sample from a subject, wherein said subject has or is suspected of having pulmonary sarcoidosis and said sample is typically a lung biopsy obtained by bronchoscopy under procedural methods not requiring general anesthesia. Gene expression analysis typically comprises determining the expression level of one or more genes.

Gene expression refers to the production of a biological product encoded by a nucleic acid sequence, such as a gene sequence. This biological product, referred to herein as a “gene product”, may be a nucleic acid or a polypeptide. The nucleic acid is typically an RNA molecule which is produced as a transcript from the gene sequence. The RNA molecule can be any type of RNA molecule, whether either before (e.g., precursor RNA) or after (e.g., mRNA) post-transcriptional processing. cDNA prepared from the mRNA of a sample is also considered a gene product. The polypeptide gene product is a peptide or protein that is encoded by the coding region of the gene, and is produced during the process of translation of the mRNA. The term “gene expression level” refers to a measure of a gene product(s) of the gene and typically refers to the relative or absolute amount or activity of the gene product. Gene expression analysis comprises determining the expression level of one or more genes.

The expression level of a gene in a sample may be determined using any appropriate method. Gene expression levels are typically determined by measuring the amount or activity of a desired gene product (i.e., an RNA or a polypeptide encoded by the coding sequence of the gene) in a biological sample. Any biological sample can be analyzed. A sample is typically a bodily tissue or fluid, such as blood, serum, plasma, urine, bone marrow, lymphatic fluid, and CNS or spinal fluid. For the purposes of the present invention the sample is typically biopsy of lung tissue obtained by bronchoscopy under procedural methods not requiring general anesthesia. A preferred procedural method is transbronchial lung biopsy (TBB). Other methods include lung mucosal biopsy, provided general anesthesia is not required.

TBB is typically performed by a pulmonologist and requires local anaesthetic to be applied to the mouth and throat. By contrast to open lung biopsy, general anesthetic is not required, the technique is considerably less invasive, and smaller tissue samples are taken. To carry out the TBB, a bronchoscope is inserted through the nose or mouth until it passes through the throat into the windpipe (trachea) and air passages of the lungs (bronchi). Samples of lung fluids may be taken through the bronchoscope. Salt water (saline) may be used to flush the area and collect cells for examination. A sample of lung tissue for the biopsy is removed using an appropriate tool, such as forceps, which is passed through the bronchoscope. Multiple samples may be collected. In some cases fluoroscopy, e.g. a chest X-ray, may be used during the procedure to help direct the forceps to the correct area of the lung. For lung mucosal biopsy, the same procedure and methods are employed as for TBB except that the forcep is targeted to the mucosa (lining of airways) visible at bronchoscopy.

Gene expression levels can be assayed qualitatively or quantitatively. The level of a gene product is measured or estimated in a sample either directly (e.g., by determining or estimating absolute level of the gene product) or relatively (e.g., by comparing the observed expression level to a gene expression level of another samples or set of samples). Measurements of gene expression levels may, but need not, include a normalization process.

Typically, mRNA levels (or cDNA prepared from such mRNA) are assayed to determine gene expression levels. Methods to detect gene expression levels include Northern blot analysis, SI nuclease mapping, quantitative polymerase chain reaction (qPCR), reverse transcription in combination with quantitative polymerase chain reaction (RT-qPCR), and reverse transcription in combination with the ligase chain reaction (RT-LCR). Multiplexed methods that allow the measurement of expression levels for more than one gene simultaneously are preferred. Measurement of gene expression levels using an oligonucleotide microarray, such as a DNA microarray, is particularly preferred. Alternative multiplexed methods include serial analysis of gene expression (SAGE) and next generation parallel and target-enrichment DNA sequencing techniques.

DNA microarrays typically consist of an arrayed series of thousands of microscopic spots of DNA oligonucleotides, called features, each containing picomoles (10⁻¹² moles) of a specific DNA sequence, known as probes. The probes are usually a short section of a gene or other DNA element that are used to hybridize a cDNA or cRNA sample (called target) under high-stringency conditions by base-pairing between complementary nucleotides. Probe-target hybridization is usually detected and quantified by detection of chemiluminescence-fluorophore-, or silver-labeled targets to determine relative abundance of nucleic acid sequences in the target. Since an array can contain tens of thousands of probes, a microarray experiment can accomplish many genetic tests in parallel. In standard microarrays, the probes are attached to a solid surface by a covalent bond to a chemical matrix (via epoxy-silane, amino-silane, lysine, polyacrylamide or others). The solid surface is typically a glass or a silicon chip. Other microarray platforms use microscopic beads, instead of the large solid support. DNA arrays are different from other types of microarray only in that they either measure DNA or use DNA as part of the detection system. Whole human genome arrays are particularly preferred, such as the Affymetrix Gene ST 1.0 array. Such arrays provide probes for whole transcript coverage, and therefore a more complete picture of gene expression, for approximately 29,000 genes from the human genome.

Alternatively or in addition, polypeptide levels can be assayed. Immunological techniques that involve antibody binding, such as enzyme linked immunosorbent assay (ELISA) and radioimmunoassay (RIA), are typically employed. Where activity assays are available, the activity of a polypeptide of interest can be assayed directly.

In the method of the invention, the observed expression level in the lung tissue biopsy for at least one group (or set) of genes is measured to determine the prognosis of pulmonary sarcoidosis in the individual subject. The at least one group of genes that is measured has been determined by evaluation typically involving a comparison between the observed gene expression level for said gene set in patients who have progressive disease (P-F) and the expression level for the same gene set in patients with self-limiting disease (N-SL). A gene set is selected for measurement in the method of the invention if said gene set is significantly differentially expressed between P-F and N-SL patients. Differential expression is determined as significant by use of statistical analyses designed for comparison of large numbers of variable. One typical method is the Bayesian moderated t-test, which is applied to identify differentially expressed genes between P-F and N-SL using the ‘limma’ (linear models for microarray analysis) package from BioConductor.

Typically, a gene set will be selected for measurement in the method of the invention where it is found that said group displays statistically significantly different expression (FDR at q=0.01 or below, more preferably q=0.001 or below) in patients with progressive disease relative to those with self-limiting disease. The gene set may typically correspond to a gene ontology-defined set of genes, such as the gene sets shown in Tables 2 and 5. When identifying a gene set of interest, particular genes within each set may be further categorised as “leading edge” genes. These are the genes which contribute most highly to the differential expression of the gene set between patients with progressive disease relative to those with self-limiting disease. The “leading edge” genes are those genes most likely to participate directly in the biological process defined by a particular gene ontology-defined set of genes.

Typically, the method of the invention may involve measurement of the expression level of at least one gene set from those listed in Table 2 or Table 5. The gene set may consist of the genes listed in Table 1 or Table 3. The method of the invention may involve measurement of the expression level of the “immune response” and/or “immune system process” and/or “lymphocyte activation” and/or “defence response” and/or “leukocyte activation” gene sets, or any combination of said gene sets.

The method may involve measurement of the expression level of at least two, three, four, five, six, seven, eight, nine, ten, twenty, thirty or all of the gene sets in Table 2.

The method may involve measurement of the expression level of at least one gene set from Table 5, more preferably at least two, three, four, five, six, seven, eight, nine, ten, twenty, thirty, forty, fifty, sixty, seventy, eighty, ninety, a hundred, or all of the gene sets in Table 5.

The gene sets in Table 5 are grouped into 10 major categories. Typically, the method of the invention may involve measurement of the expression level at least one gene set from at least one, two, three, four, five, six, seven, eight, nine, or all of the major categories shown in Table 5.

Measurement of the expression level of a gene set may typically require measurement of the expression level of each gene in that gene set. Alternatively, it may require measurement of only a representative sample of genes within a gene set. That is, the expression level of at least one, two, three, four, five, ten, twenty, thirty, forty, fifty, sixty, seventy, eighty, ninety, a hundred, or all of the genes in gene set may be measured in order to evaluate the expression level of the gene set. Alternatively, the expression level of at least 10, 20, 30, 40, 50, 60, 70, 80, 90 or 100% of the genes in a gene set may be measured in order to evaluate the expression level of the gene set. In one embodiment, only the expression level of the “leading edge” genes in a gene set will be measured in order to determine the expression level of that gene set. In one embodiment, 50%, 60%, 70%, 80%, 90% or 100% of the genes listed in Table 1 will be measured in order to determine the expression level of the gene set consisting of the genes listed in Table 1 or Table 3.

A method of the invention typically comprises categorising the sarcoidosis as either progressive or self-limiting. Typically, this method comprises:
(a) measuring the expression level at least one gene set in the sample taken from the subject;
(b) comparing the measured expression level to the expression level for the same at least one gene set as previously determined in:
   patients confirmed as having progressive sarcoidosis, and/or
   patients confirmed as having self-limiting sarcoidosis and
(c) thereby categorising the sarcoidosis in said subject as either progressive or self-limiting.

Step (c) may typically comprise categorising the sarcoidosis as:
progressive; if the expression level of the at least one gene set in the sample from the subject is significantly different to the corresponding expression level in patients confirmed as having self-limiting sarcoidosis, or if the expression level of the at least one gene set is not significantly different to the corresponding expression level in patients confirmed as having progressive sarcoidosis; or
self-limiting; if the expression level of the at least one gene set in the sample from the subject is significantly different to the corresponding expression level in patients confirmed as having progressive sarcoidosis, or if the expression level of the at least one gene set is not significantly different to the corresponding expression level in patients confirmed as having self-limiting sarcoidosis.

Where more than one gene set is measured, the above categorisations in step (c) will typically require a majority of the measured gene sets to have the significantly different or not significantly different expression levels (as appropriate) in order to categorise the sarcoidosis in a subject as either progressive or self-limiting.

### Kits

The invention further provides a test kit for use in a method for determining the prognosis of pulmonary sarcoidosis in an individual subject, which test kit comprises one or more agents suitable for analysing gene expression in a sample taken from the subject. The kit preferably comprises at least one microarray comprising a probe for each of the genes in at least one group of genes from the biologically-related categories specified above. The kit may additionally comprise one or more other reagents or instruments which enable any of the embodiments of the method mentioned above to be carried out. The kit may, optionally, comprise instructions to enable the kit to be used in the method of the invention or details regarding which individuals the method may be carried out upon.

### Therapy

The present invention also relates to the treatment of an individual identified by a method of the invention as having progressive pulmonary sarcoidosis. Thus, a substance for use in treating pulmonary sarcoidosis may be used in a method of slowing or preventing the development of pulmonary fibrosis in an individual identified by a method of the invention as having progressive pulmonary sarcoidosis. The condition of an individual identified by a method of the invention as having progressive pulmonary sarcoidosis can therefore be improved by administration of such a substance. Patient outcome can thereby be improved. A therapeutically effective amount of a substance suitable for the treatment of pulmonary fibrosis may be given to an individual identified by a method of the invention as in need thereof. Substances suitable for the treatment of pulmonary fibrosis typically include corticosteroids, new generation ‘biologics’ or immunosuppressive agents. Specific examples of corticosteroids include Prednisolone, Hydrocsortisone, and Dexamethasone. Specific examples of new generation ‘biologics’ include Rituximab and anti-TNFalpha receptor therapies such as Adalimumab, Certolizumab pegol, Golimumab and Etanercept. Specific examples of immunosuppressive agents include Methotrexate, Azathhioprine, and Mycophenolic acid.

A substance suitable for the treatment of pulmonary fibrosis is typically formulated for administration in the present invention with a pharmaceutically acceptable carrier or diluent. The pharmaceutical carrier or diluent may be, for example, an isotonic solution. For example, solid oral forms may contain, together with the active substance, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active substance, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

A therapeutically effective amount of a substance suitable for the treatment of pulmonary fibrosis is administered to a patient identified according to a method of the invention. The dose, for example of a corticosteroid, may be determined according to various parameters, especially according to the substance used; the age, weight and condition of the patient to be treated; the route of administration; and the required regimen. Again, a physician will be able to determine the required route of administration and dosage for any particular patient.

All references listed herein are incorporated herein by reference in their entireties.

The following Example illustrates the invention:

### Example 1

### Methods

### Study Population

Fifteen patients with histology-proven pulmonary sarcoidosis were recruited for the study. All patients were Caucasians, had never-smoked, were not on any inhaled or oral medications at the point of sampling, and did not present with Loefgren syndrome. Patients had only one other organ of involvement (eye or skin). All patients had active disease at the point of sampling as characterised by presence of nodularity on high-resolution thoracic CT (HRCT) scan and at least one of the following—respiratory or constitutive symptoms, peripheral lymphopenia or raised serum angiotensin-converting enzyme (ACE). In some patients, mild fibrosis was evident on thoracic HRCT. Transbronchial biopsy was performed at the point of presentation and patients were then followed up for two years. Biopsy was targeted to areas of disease (nodularity or fibro-nodularity) using CT-images. Half the sample from each patient was sent for histological analysis for granuloma and the other half (two to three biopsies) immediately transferred to RNA-later™. Patients were classified into ‘nodular, self-limiting’ (N-SL) or ‘progressive, fibrotic’ (P-F) pulmonary disease groups on the basis of persistence of symptoms and changes on CXR over two years. In the former, patients had minor symptoms (occasional cough), normal formal pulmonary function tests (FEV1, FVC and TLCO or KCO) but nodularity in typical distribution in the lung interstitium on high-resolution thoracic CT scanning; and no change in chest X ray or symptoms in the ensuing two years. In the P-F group, patients presented with fibro-nodular changes on CT scans, an abnormal spirometry or KCO, and showed persistent or progressive respiratory symptoms over two years.

The first eight patients fitting the description above were entered into the study and processed as a set (Set 1). The next eight patients fitting the same characteristics (Set 2) were processed as a second set. All patients were followed up for two years before final confirmation of groupings into N-SL or P-F.

All patients gave informed consent and the study was approved by the Oxfordshire Research Ethics Committee.

### Gene-Expression Profiling

Gene expression profiling was performed using the Affymetrix Gene ST 1.0 array which provides probes for whole transcript coverage, and therefore a more complete picture of gene expression, for approximately 29,000 genes from the human genome. Total RNA from the lung biopsies was extracted within 24 hours using the Qiagen RNeasy Mini kit according to manufacturer's instructions. RNA quality was assessed using a high-resolution electrophoresis bioanalyser system (Agilent Technologies). Isolated total RNA was then taken through standard protocols.

Raw data were processed using software (Affymetrix Power Tools) to generate RMA-normalised gene level intensities. Further analysis was performed using the R statistical program and BioConductor packages. Quality control procedures included a comparison of boxplots of RMA normalized expression values between samples, and hierarchical clustering of all samples to identify any potential outliers. Based on these and the assessment of RNA quality, 15 of the 16 samples were deemed suitable for further analysis. Raw microarray data has been deposited with GEO (Accession number GSE19976). All data collected and analyzed in the experiments adhere to the Minimal Information About a Microarray Experiment (MIAME) guidelines.

### Statistical Analysis

Prior to statistical analysis, the mean intensity was computed for each probeset and the lowest 20% were removed, leaving a total of 26,626 probesets (genes). A Bayesian moderated t-test was applied to identify differentially expressed genes between P-F and N-SL using the ‘limma’ (linear models for microarray analysis) package from BioConductor. The Benjamini-Hochberg false discovery rate (FDR) procedure was applied to correct for multiple testing. The inverse log of the absolute log 2 fold change (FC) generated by ‘limma’ was used to determine the FC between P-F and N-SL groups, with the direction of change indicated by the sign. Further visualisation of the data using heatmaps and hierarchical clustering was performed using the TMeV4 software.

Gene-set analysis methods (GSEA) are as previously described (for example by Subramaniam A et al in PNAS 2005; 102:15545-50; and Nat Genet. 2003; 34: 244-5), and were performed using web-based GSEA software, such as is available from the Broad Institute (MIT and Harvard).

qPCR validation

For quantitative real-time PCR, complementary DNA was synthesized from 200 ng DNA-free RNA using the random priming High-Capacity cDNA Archive kit (Applied Biosystems) for all patients. Quantitative rt-PCR (qPCR) was performed using the Applied Biosystems 7500 Fast Real-Time PCR system following the manufacturer's instructions. Five random genes were selected for validation using SybrGreen gene expression assays (Applied Biosystems), based on their relative expression in the groups. Two genes (TBP-TATA binding protein and HPRT—hypoxanthine phosphoribosyl-transferase 1) were chosen as endogenous controls for the normalization of all target genes as they were consistently expressed in microarray samples and not significantly different between the groups. Samples were run in triplicate along with a standard curve, to obtain reaction efficiency values. Data were then analyzed using standard methods.

### Results

### Patient Demographics

Clinical features of patients are as follows: Average age in the N-SL group was 45.1 (SD 8.5) years, and 52.0 (SD 11.6) years in P-F group; serum ACE was 64.1 (SD 10.8) and 85.1 (SD 41.5) U/1 respectively; p=0.2 (Student t test); and circulating lymphocyte count were 1.07 (SD 0.43)×10̂9/1 and 0.79 (SD 0.50)×10̂9/1 respectively, p=0.2 (Student t test). Although there was no statistical difference between the two groups for serum ACE levels and lymphocyte count; the trends in these parameters and the pulmonary function tests between the P-F and N-SL groups (worse in the P-F group) are in keeping with the clinical characteristic of symptomatic and progressive disease.

Histology of all samples (paired with samples that were submitted for RNA extraction) showed non-caseating granuloma, indicating that lung tissue around active lesions was obtained.

Quality Control of Samples from Transbronchial Biopsies

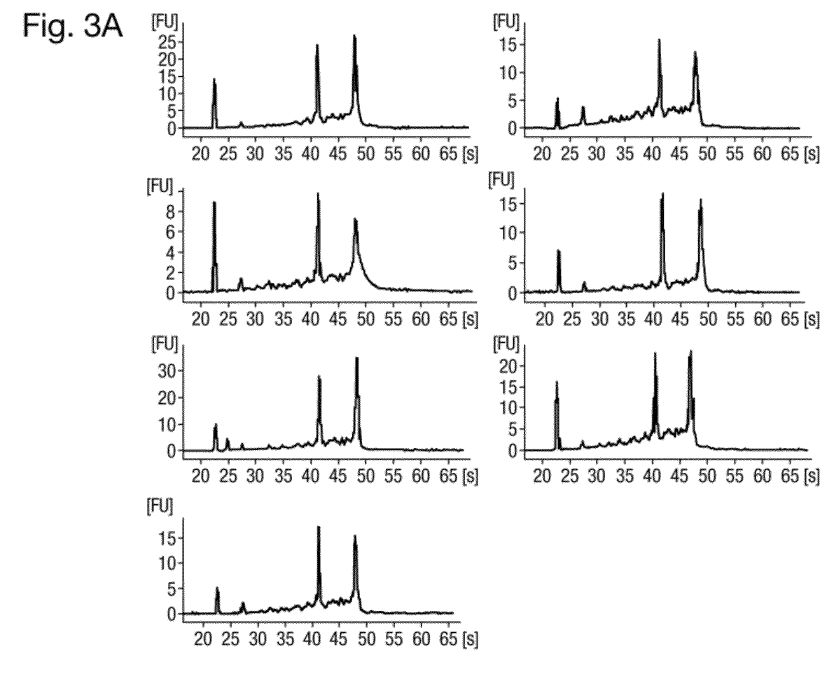

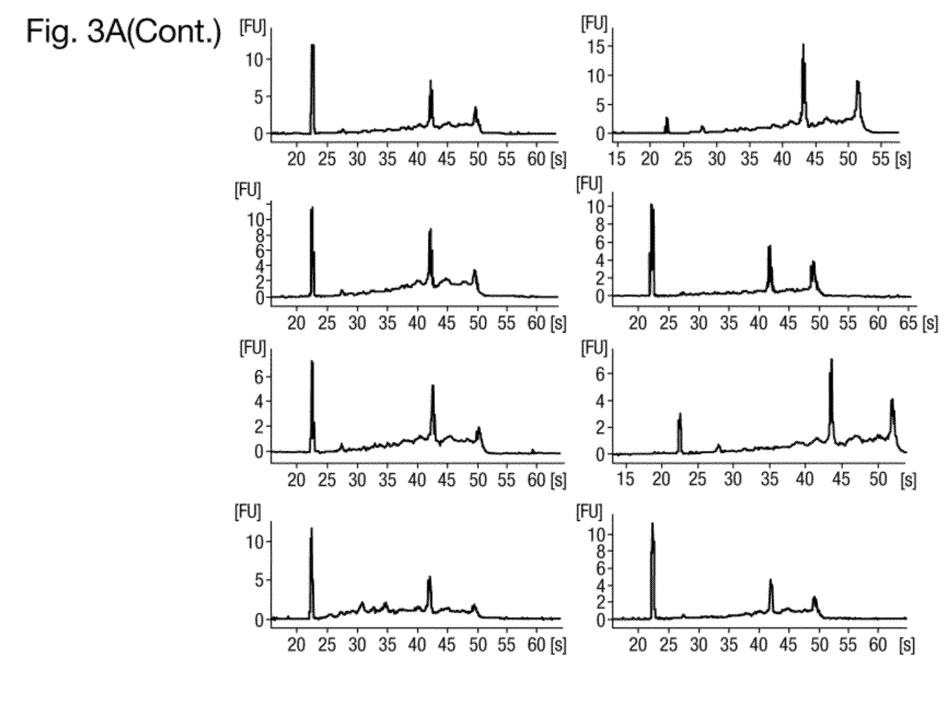

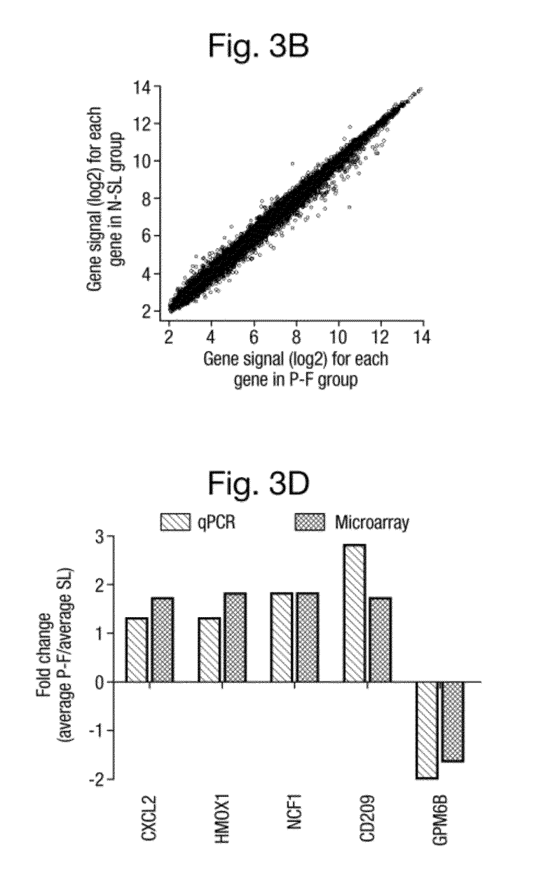

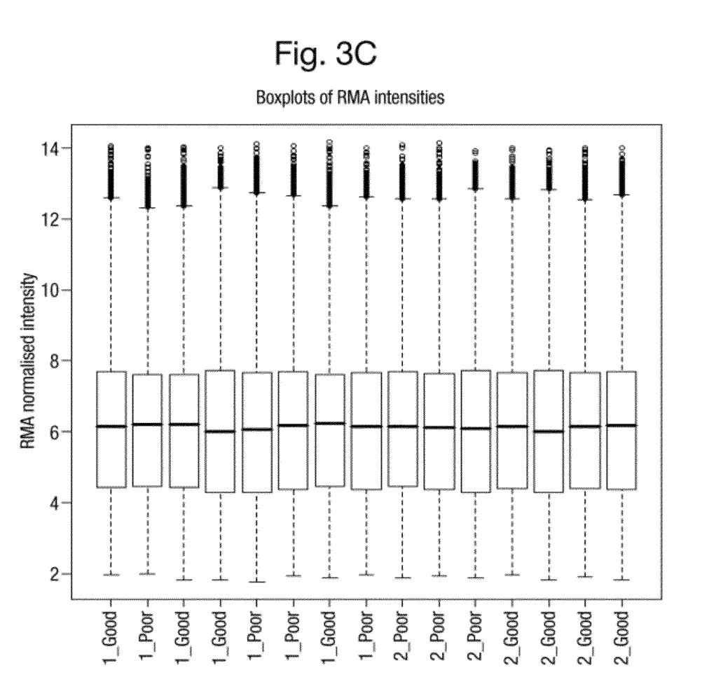

We obtained between 0.6 to 3 mcg of RNA per patient, with no evidence of degradation (FIG. 3A), or significant difference in quality between the eight biopsy samples in the first set. In the second set, one patient had a RIN value of less than 6.5 and this sample was excluded. There was a strong positive correlation between mean gene intensity in each group, as would be expected if the majority of genes are not differentially expressed (FIG. 3B), and there was no systematic difference in the distribution of fluorescence intensity on the Gene Chips used (FIG. 3C). There was also good concordance between qPCR and microarray data in the trend of change between N-SL and P-F groups for our randomly selected subset of genes (FIG. 3D).

Since there were no systematic technical difference between the two sets of patients, analyses were performed, first, on the two sets of patients independently (ie 4 N-SL vs 4 P-F patients in Set 1; and 4 N-SL vs 3 P-F in Set 2), then as a combined cohort of 8 N-SL and 7 P-F patients. Analysis of the combined dataset of 8 N-SL and 7 P-F patients is reported here; though highly similar results were obtained when the two sets of patients were considered independently.

Differentially Expressed Genes in Lungs from Self-Limiting and Progressive—Fibrotic Patient Groups.

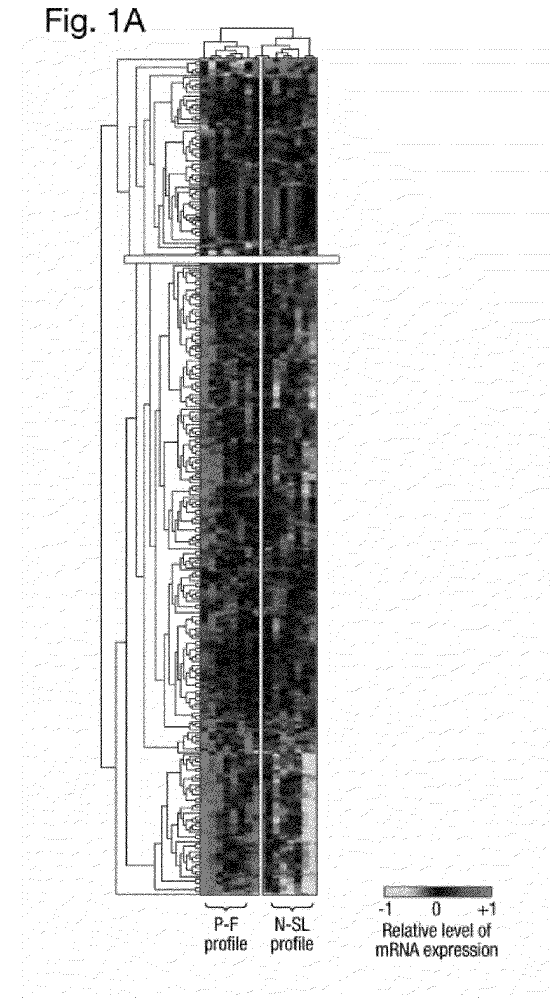

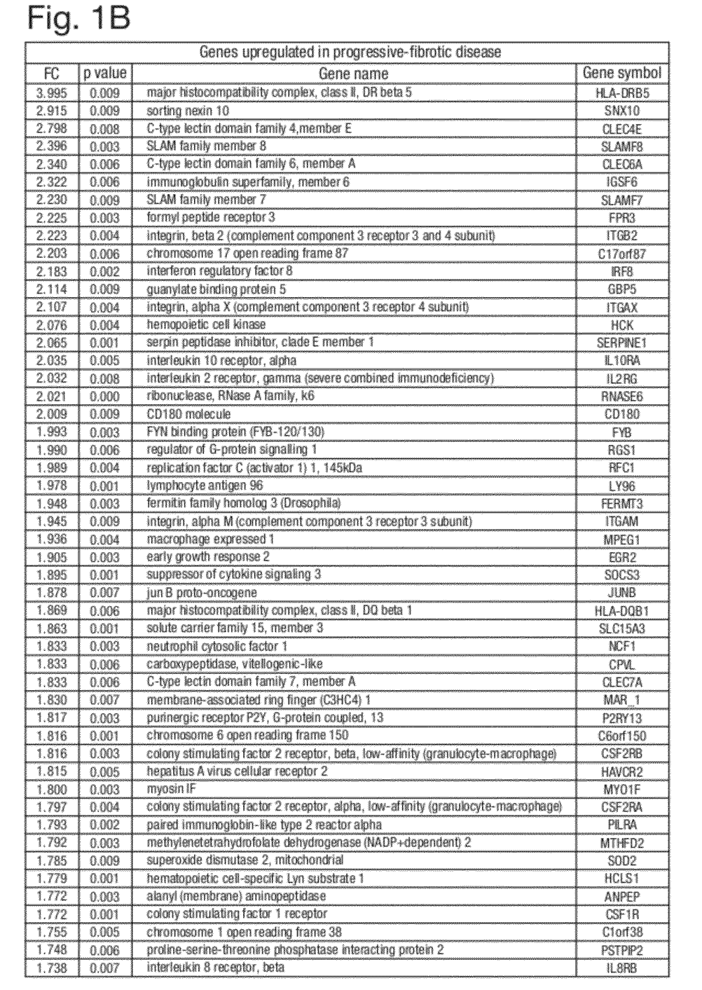

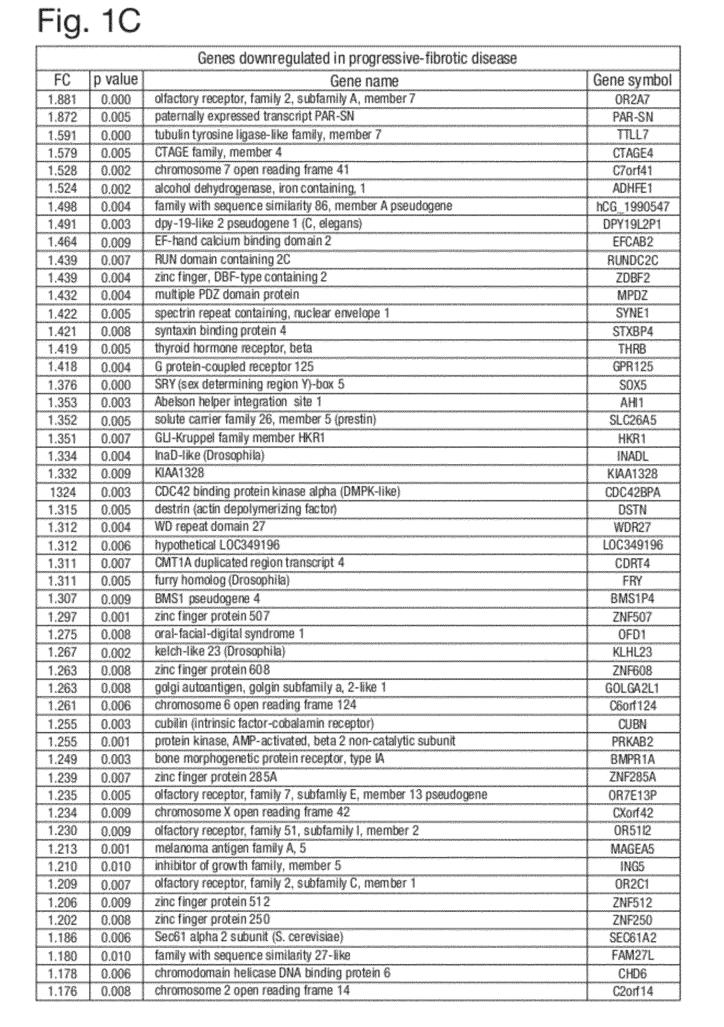

We found 334 differentially expressed genes (listed in Table 1) between the two groups of patients at a significance threshold of p<0.01 (Bayesian moderated t test, unadjusted p value; FIG. 1A). The vast majority of these genes (279) were up-regulated in the progressive-fibrotic group compared to self-limiting patients. The presence of a clear set of genes showing differential expression suggests the possibility of using these genes as part of a signature profile to discriminate between these two groups of patients and eventually to predict patients that would fall into the progressive-fibrotic group. That is, the genes listed in Table 1 represent a gene set with potential to discriminate between progressive and self-limiting sarcoidosis.

Genes over-expressed in lungs of progressive-fibrotic compared to nodular, self-limiting pulmonary sarcoidosis comprise predominantly genes related to immune activation and host defence.

Unrestricted comparisons of very large numbers of variables between two groups introduce the problem of multiple testing, where the chance of making a Type I error (falsely identifying single genes as differentially expressed) is increased. In addition, relevant sets of genes can be lost to identification because the differences are modest relative to the noise inherent to the microarray technology. One method of overcoming these issues is to compare the groups of genes using predefined, biologically meaningful sets of gene. For this, we use Gene-Set Enrichment Analysis (GSEA) to evaluate whether sets of genes, grouped together by biological processes, as defined in Gene Ontology (GO) (24,25) are enriched (over-represented) among the differentially expressed genes in the gene list generated when comparing P-F and N-SL.

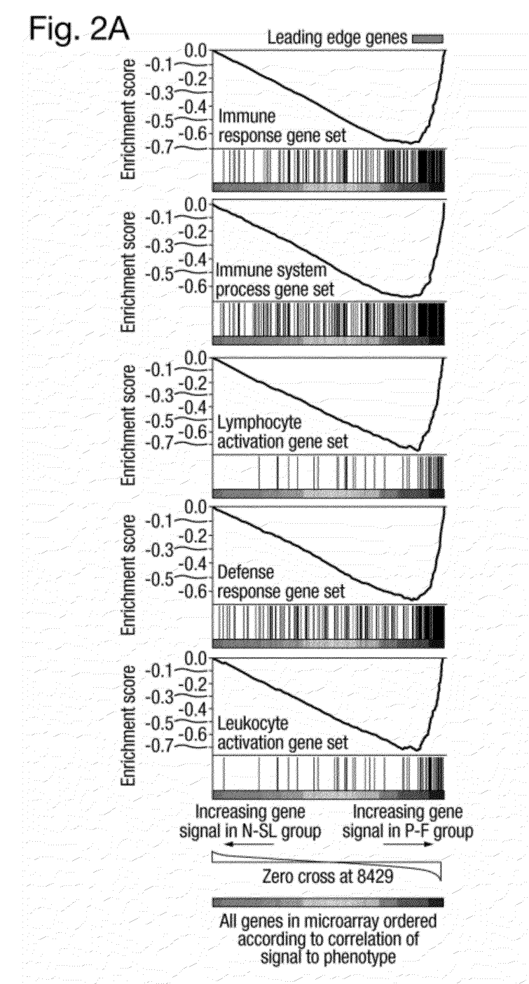

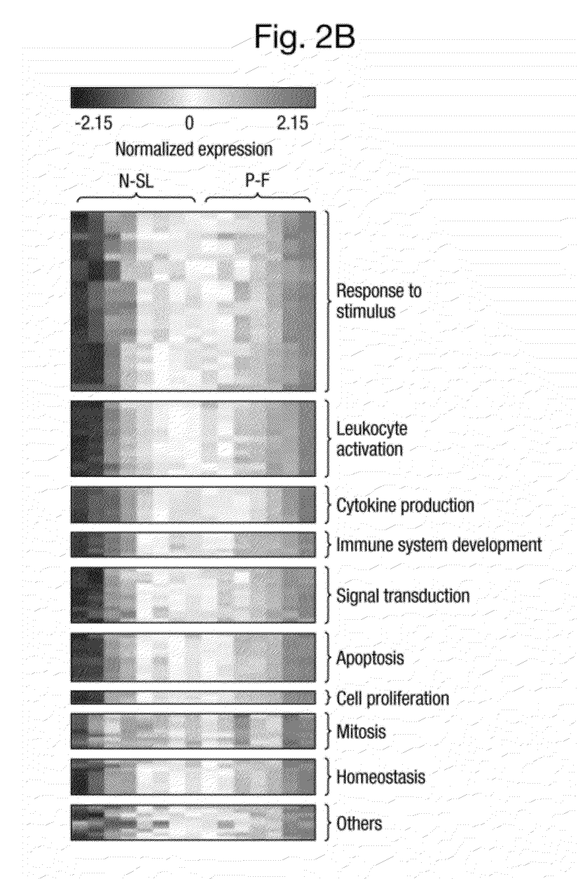

We found almost no enrichment of gene sets in genes up-regulated in the self-limiting group (only two-histone modification gene set and covalent chromatin modification gene set), but very large numbers of gene sets were highly significantly enriched among genes up-regulated in the progressive-fibrotic group [114 groups with FDR q value<0.01; and top 43 categories q<0.0001) (Table 2)]. In a higher level of evaluation, the genes within the gene-set that contribute most highly to the enrichment are extracted since these are the genes most likely to participate in the biological process within that gene-set. This subset is known as the ‘leading-edge’ genes. A graphical representation of GSEA results for the five most significant GO-based gene-sets is shown in FIG. 2A. Due to the hierarchical nature of the gene ontology, GSEA results can include several closely related categories. Based on the overlap among leading edge genes and the GO hierarchy, the 114 gene-sets with FDR q values<0.01 were grouped into 10 major categories (FIG. 2B and Table 5). This analysis showed that specific genes predominantly associated with immune activity are induced in the progressive-fibrotic group compared to the self-limiting group. This is also the case when the two sets of patients are analyzed sequentially (independent of each other). In particular, genes involved in leukocyte activation and differentiation, and cytokine production were over-expressed in P-F patients compared to the N-SL group. Other major processes that were found to be up-regulated in the P-F group included intracellular signaling (NFKB and JAK-STAT cascades) and categories related to cell life (apoptosis, cell cycle, cell proliferation and homeostasis) (FIG. 2B and Table 5). Taken together, these results suggest that immune activation is stronger in the P-F group relative to N-SL patients; and that the upregulated genes shown in FIG. 1A belong to biologically relevant gene-sets.

### DISCUSSION

This study has shown that genes upregulated in lung samples obtained from patients with progressive-fibrotic sarcoidosis, compared to self-limiting disease, comprised predominantly genes involved in host defence and immune responses. Although the number of patients in the study relatively small, (though not so for microarray studies), one strength is the reproducibility of the findings in two separate and independent studies performed sequentially.

These findings are derived from a powerful analytical approach, which assesses the differential expression of sets of biologically-relevant genes rather than individual transcripts. This is an important distinction in all studies, but particularly relevant where smaller sample sizes are involved since the signal to noise ratio of the array data may lead to false conclusions about individual gene expression values. In addition, very few genes act in isolation and complex genetic diseases like sarcoidosis are likely to be caused by groups of functionally related genes that affect immunological processes or pathways.

One potential limitation to interpretation of the data is that, although patients from both groups were bronchoscoped at active phases of their disease (the N-SL group had nodularity on HRCT and in five of the eight patients, lymphopenia), the P-F group may have been sampled at a more active point. This is consequent of our attempt to capture and compare two divergent groups of patients with different disease severity, and patients with more severe disease may also have presented with more active disease. Thus, the comparison may encapsulate both disease severity (intended) and disease activity (unintended). This could explain the upregulation of immune response genes observed in the P-F group.

In this study, we also do not have the full composition of the cellular infiltrate to the respiratory tract. Routine clinical analyses indicated that bronchoalveolar fluid from all patients showed a lymphocytosis but comprehensive flow cytometry phenotyping of cells and their proportions were not undertaken. In retrospect, this may have been a useful adjunct to the study since it would further characterise the patients and their lung disease.

We have shown is that it is possible to use standard clinical procedures to obtain high quality RNA suitable for microarray analysis from lung biopsies, and these results set a precedence and platform for translation to a clinical practice-enhancing tool.

In summary, this study provides insight into the aetio-pathology of progressive-fibrotic sarcoidosis, pointing to a persistent T cell response as the cause of chronic disease rather than genes involved in fibrogenesis. It also shows that gene array expression profiling of samples obtained using a routine clinical tool can detect a set of genes that differentiate two classes of patients with pulmonary sarcoidosis which stands up to testing using biologically relevant categories of genes.

### Tables

TABLE 1

| | | | |
|---|---|---|---|
| FC | P. Value | Gene. Title | Gene. Symbol |

Up regulated

| | | | |
|---|---|---|---|
| 3.995 | 0.009 | major histocompatibility complex, class II, DR beta 5 | HLA-DRB5 |
| 2.915 | 0.009 | sorting nexin 10 | SNX10 |
| 2.798 | 0.008 | C-type lectin domain family 4, member E | CLEC4E |
| 2.396 | 0.003 | SLAM family member 8 /// chromosome 1 open reading frame 204 | SLAMF8 /// |
| | | | C1orf204 |
| 2.340 | 0.006 | C-type lectin domain family 6, member A | CLEC6A |
| 2.322 | 0.006 | immunoglobulin superfamily, member 6 | IGSF6 |
| 2.230 | 0.009 | SLAM family member 7 | SLAMF7 |
| 2.225 | 0.003 | formyl peptide receptor 3 | FPR3 |
| 2.223 | 0.004 | integrin, beta 2 (complement component 3 receptor 3 and 4 subunit) | ITGB2 |
| 2.203 | 0.006 | chromosome 17 open reading frame 87 | C17orf87 |
| 2.183 | 0.002 | interferon regulatory factor 8 | IRF8 |
| 2.114 | 0.009 | guanylate binding protein 5 | GBP5 |
| 2.107 | 0.004 | integrin, alpha X (complement component 3 receptor 4 subunit) | ITGAX |
| 2.076 | 0.004 | hemopoietic cell kinase | HCK |
| 2.065 | 0.001 | serpin peptidase inhibitor, clade E (nexin, plasminogen activator | SERPINE1 |
| | | inhibitor type 1), member 1 | |
| 2.035 | 0.005 | interleukin 10 receptor, alpha | IL10RA |
| 2.032 | 0.008 | interleukin 2 receptor, gamma (severe combined immunodeficiency) | IL2RG |
| 2.021 | 0.000 | ribonuclease, RNase A family, k6 | RNASE6 |
| 2.009 | 0.009 | CD180 molecule | CD180 |
| 1.993 | 0.003 | FYN binding protein (FYB-120/130) | FYB |
| 1.990 | 0.006 | regulator of G-protein signaling 1 | RGS1 |
| 1.989 | 0.004 | replication factor C (activator 1) 1, 145 kDa | RFC1 |
| 1.978 | 0.001 | lymphocyte antigen 96 | LY96 |
| 1.948 | 0.003 | fermitin family homolog 3 (Drosophila) | FERMT3 |
| 1.945 | 0.009 | integrin, alpha M (complement component 3 receptor 3 subunit) | ITGAM |
| 1.936 | 0.004 | macrophage expressed 1 | MPEG1 |
| 1.905 | 0.003 | early growth response 2 | EGR2 |
| 1.895 | 0.001 | suppressor of cytokine signaling 3 | SOCS3 |
| 1.878 | 0.007 | jun B proto-oncogene | JUNB |
| 1.869 | 0.006 | major histocompatibility complex, class II, DQ beta 1 | HLA-DQB1 |
| 1.863 | 0.001 | solute carrier family 15, member 3 | SLC15A3 |
| 1.833 | 0.003 | neutrophil cytosolic factor 1 /// similar to Neutrophil cytosol factor 1 | NCF1 /// |
| | | (NCF-1) (Neutrophil NADPH oxidase factor 1) (47 kDa neutrophil | LOC648998 /// |
| | | oxidase factor) (p47-phox) (NCF-47K) (47 kDa autosomal chronic | NCF1C /// |
| | | granulomatous disease protein) (NOXO2) /// neutrophil cytosolic | NCF1B |
| | | factor 1C pseudogene /// neutrophil cytosolic factor 1B pseudogene | |
| 1.833 | 0.006 | carboxypeptidase, vitellogenic-like | CPVL |
| 1.833 | 0.006 | C-type lectin domain family 7, member A | CLEC7A |
| 1.830 | 0.007 | membrane-associated ring finger (C3HC4) 1 | Mar-01 |
| 1.817 | 0.003 | purinergic receptor P2Y, G-protein coupled, 13 | P2RY13 |
| 1.816 | 0.001 | chromosome 6 open reading frame 150 | C6orf150 |
| 1.816 | 0.003 | colony stimulating factor 2 receptor, beta, low-affinity (granulocyte- | CSF2RB |
| | | macrophage) | |
| 1.815 | 0.005 | hepatitis A virus cellular receptor 2 | HAVCR2 |
| 1.800 | 0.003 | myosin IF | MYO1F |
| 1.797 | 0.004 | colony stimulating factor 2 receptor, alpha, low-affinity (granulocyte- | CSF2RA |
| | | macrophage) | |
| 1.793 | 0.002 | paired immunoglobin-like type 2 receptor alpha | PILRA |
| 1.792 | 0.003 | methylenetetrahydrofolate dehydrogenase (NADP+ dependent) 2, | MTHFD2 |
| | | methenyltetrahydrofolate cyclohydrolase | |
| 1.785 | 0.009 | superoxide dismutase 2, mitochondrial | SOD2 |
| 1.779 | 0.001 | hematopoietic cell-specific Lyn substrate 1 | HCLS1 |
| 1.772 | 0.003 | alanyl (membrane) aminopeptidase | ANPEP |
| 1.772 | 0.001 | colony stimulating factor 1 receptor | CSF1R |
| 1.755 | 0.005 | chromosome 1 open reading frame 38 | C1orf38 |
| 1.748 | 0.006 | proline-serine-threonine phosphatase interacting protein 2 | PSTPIP2 |

Down regulated

| | | | |
|---|---|---|---|
| 1.881 | 0.000 | olfactory receptor, family 2, subfamily A, member 7 /// olfactory | OR2A7 /// |
| | | receptor, family 2, subfamily A, member 4 /// similar to rho guanine | OR2A4 /// |
| | | nucleotide exchange factor 5 | LOC728377 |
| 1.872 | 0.005 | Prader-Willi/Angelman syndrome-5 /// small nuclear | PAR5 /// SNRPN |
| | | ribonucleoprotein polypeptide N /// small nucleolar RNA, C/D box 64 | /// SNORD64 /// |
| | | /// paternally expressed transcript PAR-SN | PAR-SN |
| 1.591 | 0.000 | tubulin tyrosine ligase-like family, member 7 | TTLL7 |
| 1.579 | 0.005 | CTAGE family, member 4 /// CTAGE family, member 6 /// CTAGE | CTAGE4 /// |
| | | family member /// similar to CTAGE6 | CTAGE6 /// |
| | | | LOC100142659 |
| | | | /// LOC441294 |
| 1.528 | 0.002 | chromosome 7 open reading frame 41 | C7orf41 |
| 1.524 | 0.002 | alcohol dehydrogenase, iron containing, 1 | ADHFE1 |
| 1.498 | 0.004 | family with sequence similarity 86, member A pseudogene | hCG_1990547 |
| 1.491 | 0.003 | dpy-19-like 2 pseudogene 1 (C. elegans) | DPY19L2P1 |
| 1.464 | 0.009 | EF-hand calcium binding domain 2 | EFCAB2 |
| 1.439 | 0.007 | RUN domain containing 2C /// RUN domain containing 2B | RUNDC2C /// |
| | | | RUNDC2B |
| 1.439 | 0.004 | zinc finger, DBF-type containing 2 | ZDBF2 |
| 1.432 | 0.004 | multiple PDZ domain protein | MPDZ |
| 1.422 | 0.005 | spectrin repeat containing, nuclear envelope 1 | SYNE1 |
| 1.421 | 0.008 | syntaxin binding protein 4 | STXBP4 |
| 1.419 | 0.005 | thyroid hormone receptor, beta (erythroblastic leukemia viral (v-erb-a) | THRB |
| | | oncogene homolog 2, avian) | |
| 1.418 | 0.004 | G protein-coupled receptor 125 | GPR125 |
| 1.376 | 0.000 | SRY (sex determining region Y)-box 5 | SOX5 |
| 1.357 | 0.003 | hypothetical protein LOC100127980 | LOC100127980 |
| 1.353 | 0.003 | Abelson helper integration site 1 | AHI1 |
| 1.352 | 0.005 | solute carrier family 26, member 5 (prestin) | SLC26A5 |
| 1.351 | 0.007 | GLI-Kruppel family member HKR1 | HKR1 |
| 1.334 | 0.004 | InaD-like (Drosophila) | INADL |
| 1.332 | 0.009 | KIAA1328 | KIAA1328 |
| 1.324 | 0.003 | CDC42 binding protein kinase alpha (DMPK-like) | CDC42BPA |
| 1.315 | 0.005 | destrin (actin depolymerizing factor) | DSTN |
| 1.312 | 0.004 | WD repeat domain 27 | WDR27 |
| 1.311 | 0.007 | CMT1A duplicated region transcript 4 /// family with sequence | CDRT4 /// |
| | | similarity 18, member B2 | FAM18B2 |
| 1.311 | 0.005 | furry homolog (Drosophila) | FRY |
| 1.307 | 0.009 | BMS1 pseudogene 4 /// BMS1 pseudogene 5 /// ArfGAP with GTPase | BMS1P4 /// |
| | | domain, ankyrin repeat and PH domain 10 /// hypothetical | BMS1P5 /// |
| | | LOC399753 | AGAP10 /// |
| | | | RP11-144G6.7 |
| 1.297 | 0.001 | zinc finger protein 507 | ZNF507 |
| 1.275 | 0.008 | oral-facial-digital syndrome 1 | OFD1 |
| 1.267 | 0.002 | kelch-like 23 (Drosophila) /// phosphatase, orphan 2 | KLHL23 /// |
| | | | PHOSPHO2 |
| 1.263 | 0.008 | zinc finger protein 608 | ZNF608 |
| 1.263 | 0.008 | golgi autoantigen, golgin subfamily a, 2-like 1 | GOLGA2L1 |
| 1.261 | 0.006 | chromosome 6 open reading frame 124 | C6orf124 |
| 1.255 | 0.003 | cubilin (intrinsic factor-cobalamin receptor) | CUBN |
| 1.255 | 0.001 | protein kinase, AMP-activated, beta 2 non-catalytic subunit | PRKAB2 |
| 1.249 | 0.003 | bone morphogenetic protein receptor, type IA | BMPR1A |
| 1.239 | 0.007 | zinc finger protein 285A | ZNF285A |
| 1.235 | 0.005 | olfactory receptor, family 7, subfamily E, member 13 pseudogene | OR7E13P |
| 1.234 | 0.009 | chromosome X open reading frame 42 | CXorf42 |
| 1.230 | 0.009 | olfactory receptor, family 51, subfamily I, member 2 | OR51I2 |
| 1.220 | 0.005 | bone morphogenetic protein receptor, type IA | BMPR1A |
| 1.213 | 0.001 | melanoma antigen family A, 5 | MAGEA5 |
| 1.210 | 0.010 | inhibitor of growth family, member 5 | ING5 |
| 1.209 | 0.007 | olfactory receptor, family 2, subfamily C, member 1 | OR2C1 |
| 1.206 | 0.009 | zinc finger protein 512 | ZNF512 |
| 1.202 | 0.008 | zinc finger protein 250 | ZNF250 |
| 1.186 | 0.006 | Sec61 alpha 2 subunit (S. cerevisiae) | SEC61A2 |
| 1.180 | 0.010 | family with sequence similarity 27-like | FAM27L |
| 1.178 | 0.006 | chromodomain helicase DNA binding protein 6 | CHD6 |
| 1.176 | 0.008 | chromosome 2 open reading frame 14 /// hypothetical LOC440905 /// | C2orf14 /// |
| | | Uncharacterized protein C2orf14-like 1 /// Uncharacterized protein | LOC440905 /// |
| | | C2orf14-like 2 /// hypothetical protein LOC440910 | LOC646802 /// |
| | | | LOC100128270 |
| | | | /// LOC440910 |

TABLE 2
NAMEs of GENE SET defined by Gene Ontology

IMMUNE_SYSTEM_PROCESS
DEFENSE_RESPONSE
IMMUNE
LYMPHOCYTE_ACTIVATION
LEUKOCYTE_ACTIVATION
INFLAMMATORY_RESPONSE
T_CELL_ACTIVATION
REGULATION_OF_IMMUNE_SYSTEM_PROCESS
CELL_ACTIVATION
RESPONSE_TO_BIOTIC_STIMULUS
REGULATION_OF_LYMPHOCYTE_ACTIVATION
RESPONSE_TO_OTHER_ORGANISM
RESPONSE_TO_WOUNDING
POSITIVE_REGULATION_OF_IMMUNE_SYSTEM_PROCESS
RESPONSE_TO_EXTERNAL_STIMULUS
POSITIVE_REGULATION_OF_MULTICELLULAR_ORGANISMAL_PROCESS
CELLULAR_DEFENSE_RESPONSE
REGULATION_OF_T_CELL_ACTIVATION
RESPONSE_TO_VIRUS
MULTI_ORGANISM_PROCESS
IMMUNE_SYSTEM_DEVELOPMENT
ADAPTIVE_IMMUNE_RESPONSE
POSITIVE_REGULATION_OF_LYMPHOCYTE_ACTIVATION
HEMOPOIETIC_OR_LYMPHOID_ORGAN_DEVELOPMENT
ADAPTIVE_IMMUNE_RESPONSE_GO_0002460
HEMOPOIESIS
LEUKOCYTE_DIFFERENTIATION
CATION_HOMEOSTASIS
BEHAVIOR
I_KAPPAB_KINASE_NF_KAPPAB_CASCADE
LYMPHOCYTE_DIFFERENTIATION
POSITIVE_REGULATION_OF_T_CELL_ACTIVATION
HOMEOSTATIC_PROCESS
ION_HOMEOSTASIS
CELLULAR_CATION_HOMEOSTASIS

TABLE 3

| Gene Name | Full Gene Name | t Value | P. Value | adj. P. Val | FC |
|---|---|---|---|---|---|
| PLA2G7 | phospholipase A2, group VII (platelet- | −4.02 | 0.003 | 0.999169 | 6.21 |
| | activating factor acetylhydrolase, plasma) | | | | |
| MMP9 | matrix metallopeptidase 9 | −3.98 | 0.004 | 0.999169 | 5.36 |
| ADAMDEC1 | ADAM-like, decysin 1 | −3.67 | 0.006 | 0.999169 | 4.14 |
| LILRB4 | leukocyte immunoglobulin-like receptor, | −5.29 | 0.001 | 0.999169 | 3.57 |
| | subfamily B (with TM and ITIM domains), | | | | |
| | member 4 | | | | |
| SLAMF8 | SLAM family member 8 | −4.43 | 0.002 | 0.999169 | 3.19 |
| ITGAX | integrin, alpha X (complement component 3 | −4.72 | 0.001 | 0.999169 | 2.91 |
| | receptor 4 subunit) | | | | |
| ITGB2 | integrin, beta 2 (complement component 3 | −3.86 | 0.004 | 0.999169 | 2.84 |
| | receptor 3 and 4 subunit) | | | | |
| SLAMF7 | SLAM family member 7 | −4.29 | 0.002 | 0.999169 | 2.76 |
| FERMT3 | fermitin family homolog 3 (Drosophila) | −3.69 | 0.005 | 0.999169 | 2.55 |
| RASSF4 | Ras association (RalGDS/AF-6) domain | −3.47 | 0.008 | 0.999169 | 2.53 |
| | family member 4 | | | | |
| TMEM176A | transmembrane protein 176A | −4.11 | 0.003 | 0.999169 | 2.52 |
| APOE | apolipoprotein E | −3.57 | 0.007 | 0.999169 | 2.34 |
| C1QC | complement component 1, q subcomponent, C | −3.85 | 0.004 | 0.999169 | 2.31 |
| | chain | | | | |
| FADS1 | fatty acid desaturase 1 | −3.52 | 0.007 | 0.999169 | 2.27 |
| CAPG | capping protein (actin filament), gelsolin-like | −3.41 | 0.008 | 0.999169 | 2.27 |
| GM2A | GM2 ganglioside activator | −3.52 | 0.007 | 0.999169 | 2.23 |
| PLA2G2D | phospholipase A2, group IID | −3.39 | 0.009 | 0.999169 | 2.21 |
| CR1 | complement component (3b/4b) receptor 1 | −3.99 | 0.003 | 0.999169 | 2.19 |
| | (Knops blood group) | | | | |
| GRN | granulin | −3.51 | 0.007 | 0.999169 | 2.19 |
| MYO1F | myosin IF | −3.69 | 0.005 | 0.999169 | 2.18 |
| TNFRSF9 | tumor necrosis factor receptor superfamily, | −7.27 | 0.000 | 0.999169 | 2.17 |
| | member 9 | | | | |
| SLC15A3 | solute carrier family 15, member 3 | −3.96 | 0.004 | 0.999169 | 2.15 |
| LGMN | legumain | −3.74 | 0.005 | 0.999169 | 2.14 |
| SOD2 | superoxide dismutase 2, mitochondrial | −3.97 | 0.004 | 0.999169 | 2.13 |
| LILRB1 | leukocyte immunoglobulin-like receptor, | −4.10 | 0.003 | 0.999169 | 2.12 |
| | subfamily B (with TM and ITIM domains), | | | | |
| | member 1 | | | | |
| CYP27A1 | cytochrome P450, family 27, subfamily A, | −3.38 | 0.009 | 0.999169 | 2.05 |
| | polypeptide 1 | | | | |
| KRT7 | keratin 7 | −4.77 | 0.001 | 0.999169 | 2.01 |
| CXCL16 | chemokine (C—X—C motif) ligand 16 | −3.97 | 0.004 | 0.999169 | 2.00 |
| EMILIN2 | elastin microfibril interfacer 2 | −3.45 | 0.008 | 0.999169 | 1.99 |
| EMR2 | egf-like module containing, mucin-like, | −3.90 | 0.004 | 0.999169 | 1.98 |
| | hormone receptor-like 2 | | | | |
| UBD | ubiquitin D | −3.43 | 0.008 | 0.999169 | 1.97 |
| PILRA | paired immunoglobin-like type 2 receptor | −3.78 | 0.005 | 0.999169 | 1.97 |
| | alpha | | | | |
| TNFAIP2 | tumor necrosis factor, alpha-induced protein 2 | −4.96 | 0.001 | 0.999169 | 1.96 |
| CSF1R | colony stimulating factor 1 receptor | −4.64 | 0.001 | 0.999169 | 1.96 |
| SERPINE1 | serpin peptidase inhibitor, clade E (nexin, | −3.43 | 0.008 | 0.999169 | 1.95 |
| | plasminogen activator inhibitor type 1), | | | | |
| | member 1 | | | | |
| WARS | tryptophanyl-tRNA synthetase | −3.68 | 0.006 | 0.999169 | 1.94 |
| S100A9 | S100 calcium binding protein A9 | −3.57 | 0.007 | 0.999169 | 1.94 |
| CTSZ | cathepsin Z | −3.86 | 0.004 | 0.999169 | 1.91 |
| TAP1 | transporter 1, ATP-binding cassette, sub- | −4.20 | 0.003 | 0.999169 | 1.91 |
| | family B (MDR/TAP) | | | | |
| HMOX1 | heme oxygenase (decycling) 1 | −4.21 | 0.003 | 0.999169 | 1.91 |
| TAP2 | transporter 2, ATP-binding cassette, sub- | −4.37 | 0.002 | 0.999169 | 1.86 |
| | family B (MDR/TAP) | | | | |
| NR1H3 | nuclear receptor subfamily 1, group H, | −3.82 | 0.004 | 0.999169 | 1.86 |
| | member 3 | | | | |
| SOCS3 | suppressor of cytokine signaling 3 | −3.54 | 0.007 | 0.999169 | 1.86 |
| PIK3AP1 | phosphoinositide-3-kinase adaptor protein 1 | −3.37 | 0.009 | 0.999169 | 1.85 |
| MAN2B1 | mannosidase, alpha, class 2B, member 1 | −5.92 | 0.000 | 0.999169 | 1.84 |
| TCN2 | transcobalamin II; macrocytic anemia | −3.77 | 0.005 | 0.999169 | 1.81 |
| NCF1 | neutrophil cytosolic factor 1 | −4.00 | 0.003 | 0.999169 | 1.81 |
| IL17RA | interleukin 17 receptor A | −4.72 | 0.001 | 0.999169 | 1.80 |
| TTYH3 | tweety homolog 3 (Drosophila) | −3.91 | 0.004 | 0.999169 | 1.80 |
| C10orf64 | chromosome 10 open reading frame 64 | −3.46 | 0.008 | 0.999169 | 1.79 |
| CD4 | CD4 molecule | −4.60 | 0.001 | 0.999169 | 1.78 |
| PLAU | plasminogen activator, urokinase | −3.43 | 0.008 | 0.999169 | 1.76 |
| ARPC1B | actin related protein 2/3 complex, subunit 1B, | −3.78 | 0.005 | 0.999169 | 1.75 |
| | 41 kDa | | | | |
| IL2RA | interleukin 2 receptor, alpha | −4.15 | 0.003 | 0.999169 | 1.74 |
| TYMP | thymidine phosphorylase | −5.11 | 0.001 | 0.999169 | 1.73 |
| SCARB1 | scavenger receptor class B, member 1 | −3.38 | 0.009 | 0.999169 | 1.73 |
| WDFY4 | WDFY family member 4 | −3.62 | 0.006 | 0.999169 | 1.72 |
| KCNAB2 | potassium voltage-gated channel, shaker- | −3.95 | 0.004 | 0.999169 | 1.72 |
| | related subfamily, beta member 2 | | | | |
| CTSB | cathepsin B | −4.44 | 0.002 | 0.999169 | 1.71 |
| RND1 | Rho family GTPase 1 | −3.71 | 0.005 | 0.999169 | 1.71 |
| TPP1 | tripeptidyl peptidase I | −3.37 | 0.009 | 0.999169 | 1.71 |
| PLD3 | phospholipase D family, member 3 | −4.31 | 0.002 | 0.999169 | 1.69 |
| FGD2 | FYVE, RhoGEF and PH domain containing 2 | −3.79 | 0.005 | 0.999169 | 1.69 |
| C5orf20 | chromosome 5 open reading frame 20 | −4.10 | 0.003 | 0.999169 | 1.68 |
| PTPRJ | protein tyrosine phosphatase, receptor type, J | −4.53 | 0.002 | 0.999169 | 1.67 |
| TAPBP | TAP binding protein (tapasin) | −3.50 | 0.007 | 0.999169 | 1.65 |
| LILRB2 | leukocyte immunoglobulin-like receptor, | −3.63 | 0.006 | 0.999169 | 1.64 |
| | subfamily B (with TM and ITIM domains), | | | | |
| | member 2 | | | | |
| IL21R | interleukin 21 receptor | −5.73 | 0.000 | 0.999169 | 1.63 |
| CCNYL1 | cyclin Y-like 1 | −3.32 | 0.010 | 0.999169 | 1.62 |
| SIGLEC10 | sialic acid binding Ig-like lectin 10 | −5.31 | 0.001 | 0.999169 | 1.62 |
| ALPL | alkaline phosphatase, liver/bone/kidney | −3.40 | 0.008 | 0.999169 | 1.61 |
| NFAM1 | NFAT activating protein with ITAM motif 1 | −3.71 | 0.005 | 0.999169 | 1.60 |
| CYP27B1 | cytochrome P450, family 27, subfamily B, | −4.88 | 0.001 | 0.999169 | 1.60 |
| | polypeptide 1 | | | | |
| MAFB | v-maf musculoaponeurotic fibrosarcoma | −3.90 | 0.004 | 0.999169 | 1.60 |
| | oncogene homolog B (avian) | | | | |
| COTL1 | coactosin-like 1 (Dictyostelium) | −3.69 | 0.005 | 0.999169 | 1.59 |
| SLC37A2 | solute carrier family 37 (glycerol-3-phosphate | −3.70 | 0.005 | 0.999169 | 1.59 |
| | transporter), member 2 | | | | |
| P76 | mannose-6-phosphate protein p76 | −3.52 | 0.007 | 0.999169 | 1.59 |
| IL1R2 | interleukin 1 receptor, type II | −5.20 | 0.001 | 0.999169 | 1.58 |
| G6PD | glucose-6-phosphate dehydrogenase | −3.75 | 0.005 | 0.999169 | 1.58 |
| SLC24A6 | solute carrier family 24 | −3.36 | 0.009 | 0.999169 | 1.57 |
| | (sodium/potassium/calcium exchanger), | | | | |
| | member 6 | | | | |
| VAC14 | Vac14 homolog (S. cerevisiae) | −4.04 | 0.003 | 0.999169 | 1.57 |
| PSAP | prosaposin (variant Gaucher disease and | −3.71 | 0.005 | 0.999169 | 1.57 |
| | variant metachromatic leukodystrophy) | | | | |
| ACP2 | acid phosphatase 2, lysosomal | −4.28 | 0.002 | 0.999169 | 1.55 |
| HLA-F | major histocompatibility complex, class I, F | −3.34 | 0.009 | 0.999169 | 1.54 |
| TCIRG1 | T-cell, immune regulator 1, ATPase, H+ | −3.83 | 0.004 | 0.999169 | 1.53 |
| | transporting, lysosomal V0 subunit A3 | | | | |
| CD74 | CD74 molecule, major histocompatibility | −4.11 | 0.003 | 0.999169 | 1.53 |
| | complex, class II invariant chain | | | | |
| GPR132 | G protein-coupled receptor 132 | −3.45 | 0.008 | 0.999169 | 1.51 |
| IGF2R | insulin-like growth factor 2 receptor | −3.38 | 0.009 | 0.999169 | 1.51 |
| MMP14 | matrix metallopeptidase 14 (membrane- | −3.61 | 0.006 | 0.999169 | 1.51 |
| | inserted) | | | | |
| SRXN1 | sulfiredoxin 1 homolog (S. cerevisiae) | −3.59 | 0.006 | 0.999169 | 1.51 |
| RHBDF2 | rhomboid 5 homolog 2 (Drosophila) | −4.21 | 0.003 | 0.999169 | 1.50 |
| TRGV3 | T cell receptor gamma variable 3 | −3.56 | 0.007 | 0.999169 | 1.50 |
| PTAFR | platelet-activating factor receptor | −3.55 | 0.007 | 0.999169 | 1.50 |
| NBEAL2 | neurobeachin-like 2 | −3.32 | 0.010 | 0.999169 | 1.49 |
| IL4I1 | interleukin 4 induced 1 | −3.79 | 0.005 | 0.999169 | 1.49 |
| GNS | glucosamine (N-acetyl)-6-sulfatase | −3.30 | 0.010 | 0.999169 | 1.47 |
| | (Sanfilippo disease IIID) | | | | |
| MKI67 | antigen identified by monoclonal antibody Ki- | −4.00 | 0.003 | 0.999169 | 1.47 |
| | 67 | | | | |
| OAS3 | 2′-5′-oligoadenylate synthetase 3, 100 kDa | −3.64 | 0.006 | 0.999169 | 1.47 |
| DTX3L | deltex 3-like (Drosophila) | −3.46 | 0.008 | 0.999169 | 1.47 |
| C5AR1 | complement component 5a receptor 1 | −3.67 | 0.006 | 0.999169 | 1.46 |
| GPR68 | G protein-coupled receptor 68 | −4.06 | 0.003 | 0.999169 | 1.46 |
| MGAT1 | mannosyl (alpha-1,3-)-glycoprotein beta-1,2- | −3.98 | 0.004 | 0.999169 | 1.46 |
| | N-acetylglucosaminyltransferase | | | | |
| DPP9 | dipeptidyl-peptidase 9 | −3.51 | 0.007 | 0.999169 | 1.46 |
| FYB | FYN binding protein (FYB-120/130) | −3.58 | 0.006 | 0.999169 | 1.46 |
| PI4K2A | phosphatidylinositol 4-kinase type 2 alpha | −3.97 | 0.004 | 0.999169 | 1.45 |
| AKIRIN2 | akirin 2 | −3.41 | 0.008 | 0.999169 | 1.45 |
| LOC554223 | hypothetical LOC554223 | −3.37 | 0.009 | 0.999169 | 1.44 |
| USP6 | ubiquitin specific peptidase 6 (Tre-2 oncogene | −3.36 | 0.009 | 0.999169 | 1.44 |
| RTN1 | reticulon 1 | −3.30 | 0.010 | 0.999169 | 1.43 |
| E2F3 | E2F transcription factor 3 | −3.45 | 0.008 | 0.999169 | 1.41 |
| MIER2 | mesoderm induction early response 1, family | −3.46 | 0.008 | 0.999169 | 1.40 |
| | member 2 | | | | |
| TBC1D2 | TBC1 domain family, member 2 | −3.37 | 0.009 | 0.999169 | 1.39 |
| C19orf28 | chromosome 19 open reading frame 28 | −3.54 | 0.007 | 0.999169 | 1.39 |
| ADA | adenosine deaminase | −3.45 | 0.008 | 0.999169 | 1.38 |
| C19orf61 | chromosome 19 open reading frame 61 | −3.34 | 0.009 | 0.999169 | 1.36 |
| ECOP | EGFR-coamplified and overexpressed protein | −3.40 | 0.009 | 0.999169 | 1.35 |
| PIM1 | pim-1 oncogene | −3.32 | 0.010 | 0.999169 | 1.34 |
| MCM5 | minichromosome maintenance complex | −3.42 | 0.008 | 0.999169 | 1.33 |
| | component 5 | | | | |
| SLC25A12 | solute carrier family 25 (mitochondrial carrier, | 3.52 | 0.007 | 0.999169 | −3.64 |
| | Aralar), member 12 | | | | |
| PRG3 | proteoglycan 3 | 3.45 | 0.008 | 0.999169 | −1.72 |
| DSTN | destrin (actin depolymerizing factor) | 3.41 | 0.008 | 0.999169 | −1.70 |
| SOX5 | SRY (sex determining region Y)-box 5 | 4.07 | 0.003 | 0.999169 | −1.65 |
| TBC1D8 | TBC1 domain family, member 8 | 3.37 | 0.009 | 0.999169 | −1.60 |
| LOC349196 | hypothetical LOC349196 | 3.37 | 0.009 | 0.999169 | −1.56 |
| METT5D1 | methyltransferase 5 domain containing 1 | 3.80 | 0.005 | 0.999169 | −1.53 |
| DHX35 | DEAH (Asp-Glu-Ala-His) box polypeptide 35 | 3.36 | 0.009 | 0.999169 | −1.48 |
| LOC134466 | hypothetical protein LOC134466 | 3.47 | 0.008 | 0.999169 | −1.48 |
| LOC100127980 | hypothetical protein LOC100127980 | 4.75 | 0.001 | 0.999169 | −1.46 |
| RP4-621O15.2 | hypothetical protein FLJ31401 | 3.49 | 0.007 | 0.999169 | −1.46 |
| TTLL7 | tubulin tyrosine ligase-like family, member 7 | 4.02 | 0.003 | 0.999169 | −1.43 |
| DPY19L2 | dpy-19-like 2 (C. elegans) | 3.33 | 0.009 | 0.999169 | −1.43 |
| NEGR1 | neuronal growth regulator 1 | 3.57 | 0.007 | 0.999169 | −1.34 |
| PRUNE2 | prune homolog 2 (Drosophila) | 3.32 | 0.010 | 0.999169 | −1.34 |

TABLE 4

| | Size of | | FDR |
|---|---|---|---|
| Gene ontology-defined biological categories | gene set | NES | q-val |
| IMMUNE_SYSTEM_PROCESS | 275 | −2.75 | 0 |
| IMMUNE_RESPONSE | 192 | −2.75 | 0 |
| DEFENSE_RESPONSE | 203 | −2.66 | 0 |
| LOCOMOTORY_BEHAVIOR | 77 | −2.61 | 0 |
| INFLAMMATORY_RESPONSE | 101 | −2.55 | 0 |
| RESPONSE_TO_EXTERNAL_STIMULUS | 236 | −2.42 | 0 |
| RESPONSE_TO_WOUNDING | 149 | −2.41 | 0 |
| BEHAVIOR | 112 | −2.40 | 0 |
| LYMPHOCYTE_ACTIVATION | 51 | −2.34 | 0 |
| CELLULAR_DEFENSE_RESPONSE | 44 | −2.33 | 0 |
| MULTI_ORGANISM_PROCESS | 113 | −2.29 | 0 |
| CELL_ACTIVATION | 64 | −2.25 | 2.0E−04 |
| RESPONSE_TO_BIOTIC_STIMULUS | 95 | −2.25 | 1.9E−04 |
| RESPONSE_TO_OTHER_ORGANISM | 61 | −2.24 | 1.7E−04 |
| HEMOPOIETIC_OR_LYMPHOID_ORGAN_DEVELOPMENT | 67 | −2.24 | 1.6E−04 |
| IMMUNE_SYSTEM_DEVELOPMENT | 70 | −2.24 | 1.5E−04 |
| HEMOPOIESIS | 66 | −2.23 | 1.4E−04 |
| LEUKOCYTE_ACTIVATION | 58 | −2.23 | 1.4E−04 |
| RAS_PROTEIN_SIGNAL_TRANSDUCTION | 61 | −2.22 | 1.3E−04 |
| T_CELL_ACTIVATION | 36 | −2.20 | 1.2E−04 |
| RESPONSE_TO_VIRUS | 37 | −2.18 | 1.2E−04 |
| PROTEIN_KINASE_CASCADE | 257 | −2.16 | 1.6E−04 |
| REGULATION_OF_DEVELOPMENTAL_PROCESS | 378 | −2.15 | 1.6E−04 |
| REGULATION_OF_PROGRAMMED_CELL_DEATH | 302 | −2.14 | 2.0E−04 |
| LIPID_TRANSPORT | 25 | −2.13 | 2.0E−04 |
| INTRACELLULAR_SIGNALING_CASCADE | 568 | −2.13 | 1.9E−04 |
| HOMEOSTATIC_PROCESS | 169 | −2.13 | 1.8E−04 |
| REGULATION_OF_APOPTOSIS | 301 | −2.13 | 1.8E−04 |
| REGULATION_OF_SIGNAL_TRANSDUCTION | 190 | −2.12 | 2.1E−04 |
| POSITIVE_REGULATION_OF_IMMUNE_SYSTEM_PROCESS | 42 | −2.11 | 2.0E−04 |
| REGULATION_OF_IMMUNE_SYSTEM_PROCESS | 54 | −2.10 | 2.0E−04 |
| POSITIVE_REGULATION_OF_SIGNAL_TRANSDUCTION | 108 | −2.10 | 1.9E−04 |
| SMALL_GTPASE_MEDIATED_SIGNAL_TRANSDUCTION | 80 | −2.10 | 2.2E−04 |
| I_KAPPAB_KINASE_NF_KAPPAB_CASCADE | 101 | −2.08 | 2.9E−04 |
| RESPONSE_TO_CHEMICAL_STIMULUS | 252 | −2.08 | 2.8E−04 |
| CHEMICAL_HOMEOSTASIS | 121 | −2.07 | 2.8E−04 |
| REGULATION_OF_I_KAPPAB_KINASE_NF_KAPPAB_CASCADE | 82 | −2.06 | 3.3E−04 |
| REGULATION_OF_HYDROLASE_ACTIVITY | 68 | −2.04 | 4.9E−04 |
| PROGRAMMED_CELL_DEATH | 386 | −2.04 | 5.1E−04 |
| LEUKOCYTE_DIFFERENTIATION | 32 | −2.04 | 4.9E−04 |
| POSITIVE_REGULATION_OF_I_KAPPAB_KINASE_NF_KAPPAB_CASCADE | 77 | −2.04 | 4.8E−04 |
| APOPTOSIS_GO | 385 | −2.04 | 4.7E−04 |
| NEGATIVE_REGULATION_OF_DEVELOPMENTAL_PROCESS | 161 | −2.03 | 5.7E−04 |
| POSITIVE_REGULATION_OF_CELL_PROLIFERATION | 111 | −2.03 | 5.9E−04 |
| CATION_HOMEOSTASIS | 84 | −2.03 | 5.7E−04 |
| REGULATION_OF_BIOLOGICAL_QUALITY | 334 | −2.03 | 5.6E−04 |
| RHO_PROTEIN_SIGNAL_TRANSDUCTION | 36 | −2.03 | 5.5E−04 |
| ION_HOMEOSTASIS | 98 | −2.02 | 5.6E−04 |
| CELL_PROLIFERATION_GO_0008283 | 427 | −2.01 | 5.5E−04 |
| POSITIVE_REGULATION_OF_BIOLOGICAL_PROCESS | 599 | −2.01 | 7.4E−04 |
| JAK_STAT_CASCADE | 27 | −2.01 | 7.5E−04 |
| ADAPTIVE_IMMUNE_RESPONSE | 21 | −2.00 | 7.5E−04 |
| POSITIVE_REGULATION_OF_HYDROLASE_ACTIVITY | 44 | −2.00 | 7.6E−04 |
| CYTOKINE_PRODUCTION | 59 | −2.00 | 7.5E−04 |
| REGULATION_OF_ANATOMICAL_STRUCTURE_MORPHOGENESIS | 24 | −1.99 | 8.5E−04 |
| ADAPTIVE_IMMUNE_RESPONSE_GO_0002460 | 20 | −1.99 | 8.5E−04 |
| REGULATION_OF_LYMPHOCYTE_ACTIVATION | 29 | −1.99 | 8.8E−04 |
| POSITIVE_REGULATION_OF_CELLULAR_PROCESS | 563 | −1.99 | 8.9E−04 |
| NEGATIVE_REGULATION_OF_APOPTOSIS | 128 | −1.98 | 9.0E−04 |
| CELLULAR_CATION_HOMEOSTASIS | 81 | −1.97 | 0.001 |
| NEGATIVE_REGULATION_OF_PROGRAMMED_CELL_DEATH | 129 | −1.97 | 0.001 |
| HOMEOSTASIS_OF_NUMBER_OF_CELLS | 20 | −1.96 | 0.001 |
| LYMPHOCYTE_DIFFERENTIATION | 22 | −1.96 | 0.001 |
| REGULATION_OF_IMMUNE_RESPONSE | 28 | −1.96 | 0.001 |
| POSITIVE_REGULATION_OF_JNK_ACTIVITY | 18 | −1.95 | 0.001 |
| POSITIVE_REGULATION_OF_RESPONSE_TO_STIMULUS | 34 | −1.94 | 0.001 |
| CYTOKINE_AND_CHEMOKINE_MEDIATED_SIGNALING_PATHWAY | 17 | −1.94 | 0.002 |
| REGULATION_OF_CELL_ADHESION | 27 | −1.93 | 0.002 |
| CELL_DEVELOPMENT | 503 | −1.93 | 0.002 |
| PEPTIDYL_TYROSINE_MODIFICATION | 23 | −1.93 | 0.002 |
| HUMORAL_IMMUNE_RESPONSE | 29 | −1.93 | 0.002 |
| POSITIVE_REGULATION_OF_MULTICELLULAR_ORGANISMAL_PROCESS | 56 | −1.93 | 0.002 |
| CELL_SURFACE_RECEPTOR_LINKED_SIGNAL_TRANSDUCTION_GO_0007166 | 477 | −1.92 | 0.002 |
| PEPTIDYL_TYROSINE_PHOSPHORYLATION | 21 | −1.92 | 0.002 |
| REGULATION_OF_MOLECULAR_FUNCTION | 278 | −1.92 | 0.002 |
| REGULATION_OF_CATALYTIC_ACTIVITY | 235 | −1.92 | 0.002 |
| CYTOKINE_METABOLIC_PROCESS | 33 | −1.92 | 0.002 |
| MYELOID_CELL_DIFFERENTIATION | 33 | −1.91 | 0.002 |
| APOPTOTIC_PROGRAM | 52 | −1.91 | 0.002 |
| POSITIVE_REGULATION_OF_CASPASE_ACTIVITY | 25 | −1.91 | 0.002 |
| ANTI_APOPTOSIS | 103 | −1.91 | 0.002 |
| POSITIVE_REGULATION_OF_PROTEIN_METABOLIC_PROCESS | 60 | −1.90 | 0.002 |
| POSITIVE_REGULATION_OF_IMMUNE_RESPONSE | 25 | −1.89 | 0.002 |
| MEMBRANE_ORGANIZATION_AND_BIOGENESIS | 126 | −1.89 | 0.002 |
| REGULATION_OF_CELL_PROLIFERATION | 252 | −1.89 | 0.002 |
| POSITIVE_REGULATION_OF_CELLULAR_PROTEIN_METABOLIC_PROCESS | 58 | −1.89 | 0.002 |
| CYTOKINE_BIOSYNTHETIC_PROCESS | 32 | −1.88 | 0.003 |
| RESPONSE_TO_BACTERIUM | 21 | −1.88 | 0.003 |
| REGULATION_OF_CELL_MORPHOGENESIS | 15 | −1.88 | 0.003 |
| PROTEIN_AMINO_ACID_PHOSPHORYLATION | 246 | −1.86 | 0.003 |
| POSITIVE_REGULATION_OF_CATALYTIC_ACTIVITY | 137 | −1.86 | 0.003 |
| ACTIVATION_OF_JNK_ACTIVITY | 16 | −1.85 | 0.004 |
| POSITIVE_REGULATION_OF_LYMPHOCYTE_ACTIVATION | 20 | −1.85 | 0.004 |
| LIPID_CATABOLIC_PROCESS | 28 | −1.85 | 0.004 |
| PROTEOLYSIS | 168 | −1.85 | 0.004 |
| REGULATION_OF_RESPONSE_TO_STIMULUS | 42 | −1.85 | 0.004 |
| CELLULAR_LIPID_CATABOLIC_PROCESS | 27 | −1.85 | 0.004 |
| RESPONSE_TO_STRESS | 432 | −1.84 | 0.004 |
| MITOTIC_CELL_CYCLE | 140 | −1.84 | 0.004 |
| CELLULAR_HOMEOSTASIS | 112 | −1.83 | 0.005 |
| BLOOD_COAGULATION | 34 | −1.82 | 0.006 |
| REGULATION_OF_CYTOKINE_BIOSYNTHETIC_PROCESS | 30 | −1.81 | 0.006 |
| B_CELL_ACTIVATION | 17 | −1.81 | 0.006 |
| PHOSPHORYLATION | 273 | −1.81 | 0.006 |
| RECEPTOR_MEDIATED_ENDOCYTOSIS | 32 | −1.80 | 0.006 |
| COAGULATION | 35 | −1.80 | 0.007 |
| CARBOXYLIC_ACID_METABOLIC_PROCESS | 148 | −1.80 | 0.006 |
| T_CELL_PROLIFERATION | 15 | −1.80 | 0.006 |
| ORGANIC_ACID_METABOLIC_PROCESS | 150 | −1.80 | 0.006 |
| REGULATION_OF_T_CELL_ACTIVATION | 23 | −1.80 | 0.006 |
| CASPASE_ACTIVATION | 21 | −1.80 | 0.007 |
| VIRAL_REPRODUCTIVE_PROCESS | 32 | −1.79 | 0.007 |
| REGULATION_OF_KINASE_ACTIVITY | 136 | −1.79 | 0.007 |
| REGULATION_OF_TRANSFERASE_ACTIVITY | 139 | −1.79 | 0.007 |
| M_PHASE_OF_MITOTIC_CELL_CYCLE | 76 | −1.79 | 0.007 |
| CYTOKINESIS | 15 | −1.79 | 0.007 |
| ACTIN_CYTOSKELETON_ORGANIZATION_AND_BIOGENESIS | 96 | −1.78 | 0.007 |
| MITOSIS | 74 | −1.78 | 0.007 |
| REGULATION_OF_PROTEIN_KINASE_ACTIVITY | 134 | −1.78 | 0.007 |
| POSITIVE_REGULATION_OF_TRANSFERASE_ACTIVITY | 72 | −1.78 | 0.008 |
| POSITIVE_REGULATION_OF_TRANSLATION | 27 | −1.77 | 0.008 |
| ACTIVATION_OF_MAPK_ACTIVITY | 34 | −1.77 | 0.008 |
| PROTEIN_AMINO_ACID_DEPHOSPHORYLATION | 55 | −1.77 | 0.008 |
| PROTEIN_COMPLEX_ASSEMBLY | 152 | −1.77 | 0.008 |
| NEGATIVE_REGULATION_OF_MULTICELLULAR_ORGANISMAL_PROCESS | 22 | −1.77 | 0.008 |
| PIGMENT_BIOSYNTHETIC_PROCESS | 16 | −1.77 | 0.008 |
| REGULATION_OF_JNK_ACTIVITY | 20 | −1.76 | 0.009 |
| DEPHOSPHORYLATION | 62 | −1.76 | 0.009 |
| IMMUNE_EFFECTOR_PROCESS | 30 | −1.76 | 0.009 |
| ACTIN_FILAMENT_BASED_PROCESS | 104 | −1.76 | 0.009 |
| G1_S_TRANSITION_OF_MITOTIC_CELL_CYCLE | 24 | −1.75 | 0.009 |
| WOUND_HEALING | 41 | −1.75 | 0.009 |
| REGULATION_OF_MITOSIS | 35 | −1.75 | 0.009 |
| POSITIVE_REGULATION_OF_DEVELOPMENTAL_PROCESS | 191 | −1.75 | 0.009 |
| HEMOSTASIS | 36 | −1.74 | 0.011 |
| REGULATION_OF_MULTICELLULAR_ORGANISMAL_PROCESS | 116 | −1.73 | 0.011 |
| POSITIVE_REGULATION_OF_T_CELL_ACTIVATION | 18 | −1.73 | 0.011 |
| INDUCTION_OF_APOPTOSIS_BY_EXTRACELLULAR_SIGNALS | 24 | −1.73 | 0.011 |
| AMINE_METABOLIC_PROCESS | 122 | −1.73 | 0.011 |
| POSITIVE_REGULATION_OF_MAP_KINASE_ACTIVITY | 40 | −1.73 | 0.011 |
| ACTIVATION_OF_IMMUNE_RESPONSE | 17 | −1.73 | 0.012 |
| VIRAL_REPRODUCTION | 36 | −1.72 | 0.012 |
| LIPID_METABOLIC_PROCESS | 261 | −1.72 | 0.012 |
| CELLULAR_CARBOHYDRATE_CATABOLIC_PROCESS | 17 | −1.71 | 0.013 |
| AMINO_ACID_METABOLIC_PROCESS | 65 | −1.70 | 0.014 |
| CELL_CYCLE_PHASE | 151 | −1.69 | 0.015 |
| DEFENSE_RESPONSE_TO_BACTERIUM | 15 | −1.69 | 0.016 |
| VIRAL_INFECTIOUS_CYCLE | 28 | −1.69 | 0.016 |
| NEGATIVE_REGULATION_OF_BIOLOGICAL_PROCESS | 586 | −1.68 | 0.016 |
| CARBOHYDRATE_CATABOLIC_PROCESS | 17 | −1.68 | 0.016 |
| REGULATION_OF_PROTEIN_MODIFICATION_PROCESS | 36 | −1.68 | 0.016 |
| REGULATION_OF_BODY_FLUID_LEVELS | 40 | −1.68 | 0.016 |
| INNATE_IMMUNE_RESPONSE | 15 | −1.68 | 0.016 |
| NITROGEN_COMPOUND_METABOLIC_PROCESS | 135 | −1.68 | 0.016 |
| MAPKKK_CASCADE_GO_0000165 | 90 | −1.68 | 0.016 |
| ORGAN_DEVELOPMENT | 437 | −1.68 | 0.017 |
| VASCULATURE_DEVELOPMENT | 41 | −1.68 | 0.017 |
| ORGAN_MORPHOGENESIS | 105 | −1.68 | 0.017 |
| PIGMENT_METABOLIC_PROCESS | 17 | −1.67 | 0.017 |
| MITOTIC_CELL_CYCLE_CHECKPOINT | 20 | −1.67 | 0.017 |
| CELL_CYCLE_PROCESS | 174 | −1.67 | 0.018 |
| REGULATION_OF_PROTEIN_AMINO_ACID_PHOSPHORYLATION | 22 | −1.66 | 0.019 |
| ACTIN_FILAMENT_ORGANIZATION | 22 | −1.66 | 0.019 |
| REGULATION_OF_MAP_KINASE_ACTIVITY | 58 | −1.66 | 0.019 |
| AMINE_TRANSPORT | 31 | −1.65 | 0.020 |
| POST_TRANSLATIONAL_PROTEIN_MODIFICATION | 420 | −1.65 | 0.021 |
| ANGIOGENESIS | 34 | −1.65 | 0.021 |
| CELLULAR_CATABOLIC_PROCESS | 181 | −1.65 | 0.021 |
| CELLULAR_PROTEIN_COMPLEX_ASSEMBLY | 31 | −1.64 | 0.022 |
| NEGATIVE_REGULATION_OF_CELLULAR_PROCESS | 569 | −1.64 | 0.022 |
| STEROID_METABOLIC_PROCESS | 50 | −1.64 | 0.022 |
| RESPONSE_TO_DRUG | 20 | −1.64 | 0.023 |
| CELLULAR_LIPID_METABOLIC_PROCESS | 205 | −1.64 | 0.023 |
| REGULATION_OF_PROTEIN_METABOLIC_PROCESS | 150 | −1.64 | 0.023 |
| INTERACTION_WITH_HOST | 16 | −1.63 | 0.023 |
| POSITIVE_REGULATION_OF_CYTOKINE_BIOSYNTHETIC_PROCESS | 21 | −1.63 | 0.024 |
| CATABOLIC_PROCESS | 191 | −1.63 | 0.024 |
| HETEROCYCLE_METABOLIC_PROCESS | 26 | −1.62 | 0.025 |
| CYTOSKELETON_ORGANIZATION_AND_BIOGENESIS | 189 | −1.62 | 0.026 |
| G_PROTEIN_COUPLED_RECEPTOR_PROTEIN_SIGNALING_PATHWAY | 227 | −1.61 | 0.027 |
| REGULATION_OF_CELL_MIGRATION | 24 | −1.61 | 0.027 |
| ESTABLISHMENT_OF_ORGANELLE_LOCALIZATION | 16 | −1.61 | 0.027 |
| MONOCARBOXYLIC_ACID_METABOLIC_PROCESS | 71 | −1.61 | 0.027 |
| CELL_DIVISION | 17 | −1.61 | 0.027 |
| VIRAL_GENOME_REPLICATION | 17 | −1.61 | 0.027 |
| NEGATIVE_REGULATION_OF_CATALYTIC_ACTIVITY | 58 | −1.61 | 0.027 |
| REGULATION_OF_RAS_PROTEIN_SIGNAL_TRANSDUCTION | 18 | −1.60 | 0.029 |
| REGULATION_OF_CELL_CYCLE | 163 | −1.59 | 0.031 |
| POSITIVE_REGULATION_OF_PROTEIN_MODIFICATION_PROCESS | 22 | −1.59 | 0.032 |
| CELL_CYCLE_GO_0007049 | 284 | −1.59 | 0.032 |
| REGULATION_OF_CELLULAR_PROTEIN_METABOLIC_PROCESS | 141 | −1.58 | 0.033 |
| REGULATION_OF_PHOSPHORYLATION | 36 | −1.58 | 0.033 |
| M_PHASE | 98 | −1.58 | 0.033 |
| CARBOHYDRATE_METABOLIC_PROCESS | 148 | −1.58 | 0.035 |
| REGULATION_OF_DNA_BINDING | 40 | −1.57 | 0.037 |
| MAINTENANCE_OF_LOCALIZATION | 18 | −1.56 | 0.038 |
| AMINO_ACID_TRANSPORT | 22 | −1.56 | 0.040 |
| PROTEIN_SECRETION | 24 | −1.55 | 0.043 |
| CELLULAR_CARBOHYDRATE_METABOLIC_PROCESS | 108 | −1.55 | 0.044 |
| POSITIVE_REGULATION_OF_CELLULAR_METABOLIC_PROCESS | 192 | −1.54 | 0.044 |
| REGULATION_OF_GTPASE_ACTIVITY | 16 | −1.54 | 0.044 |
| SPHINGOLIPID_METABOLIC_PROCESS | 22 | −1.54 | 0.044 |
| CELL_MIGRATION | 79 | −1.54 | 0.044 |
| ORGANELLE_LOCALIZATION | 23 | −1.53 | 0.047 |
| POSITIVE_REGULATION_OF_CELLULAR_COMPONENT_ORGANIZATION_AND_BIOGENESIS | 30 | −1.53 | 0.047 |
| CELLULAR_RESPONSE_TO_STIMULUS | 16 | −1.53 | 0.049 |
| TISSUE_DEVELOPMENT | 103 | −1.53 | 0.049 |
| POSITIVE_REGULATION_OF_METABOLIC_PROCESS | 198 | −1.53 | 0.049 |
| MESODERM_DEVELOPMENT | 22 | −1.52 | 0.050 |
| VESICLE_MEDIATED_TRANSPORT | 178 | −1.52 | 0.050 |
| PROTEIN_OLIGOMERIZATION | 33 | −1.52 | 0.050 |
| CELLULAR_MACROMOLECULE_CATABOLIC_PROCESS | 93 | −1.51 | 0.052 |
| COFACTOR_METABOLIC_PROCESS | 50 | −1.51 | 0.053 |
| INTERPHASE | 63 | −1.51 | 0.053 |
| SECONDARY_METABOLIC_PROCESS | 22 | −1.51 | 0.053 |
| REGULATION_OF_CELL_GROWTH | 37 | −1.51 | 0.053 |
| CHROMOSOME_SEGREGATION | 29 | −1.51 | 0.054 |
| NEGATIVE_REGULATION_OF_CELL_PROLIFERATION | 132 | −1.50 | 0.056 |
| MEMBRANE_LIPID_METABOLIC_PROCESS | 87 | −1.50 | 0.056 |
| NEGATIVE_REGULATION_OF_TRANSFERASE_ACTIVITY | 31 | −1.50 | 0.057 |
| PROTEIN_TRANSPORT | 147 | −1.50 | 0.056 |
| PEPTIDYL_AMINO_ACID_MODIFICATION | 55 | −1.50 | 0.057 |
| REGULATION_OF_SMALL_GTPASE_MEDIATED_SIGNAL_TRANSD^{N} | 22 | −1.50 | 0.057 |
| POSITIVE_REGULATION_OF_PHOSPHATE_METABOLIC_PROCESS | 18 | −1.50 | 0.057 |
| SECOND_MESSENGER_MEDIATED_SIGNALING | 111 | −1.50 | 0.057 |
| REGULATION_OF_PROTEIN_SECRETION | 16 | −1.49 | 0.059 |
| MITOCHONDRION_ORGANIZATION_AND_BIOGENESIS | 44 | −1.49 | 0.060 |
| RESPONSE_TO_EXTRACELLULAR_STIMULUS | 22 | −1.49 | 0.061 |
| PROTEIN_IMPORT_INTO_NUCLEUS | 44 | −1.49 | 0.061 |
| PROTEIN_LOCALIZATION | 194 | −1.48 | 0.061 |
| REGULATION_OF_BINDING | 51 | −1.48 | 0.062 |
| POSITIVE_REGULATION_OF_DNA_BINDING | 21 | −1.48 | 0.062 |
| BIOPOLYMER_MODIFICATION | 578 | −1.48 | 0.063 |
| PROTEIN_MODIFICATION_PROCESS | 562 | −1.48 | 0.064 |
| ESTABLISHMENT_OF_CELLULAR_LOCALIZATION | 323 | −1.47 | 0.064 |
| STRESS_ACTIVATED_PROTEIN_KINASE_SIGNALING_PATHWAY | 47 | −1.47 | 0.064 |
| REGULATION_OF_G_PROTEIN_COUPLED_RECEPTOR_PROTEIN_SIGNALING_PATHWAY | 18 | −1.47 | 0.065 |
| NEGATIVE_REGULATION_OF_GROWTH | 34 | −1.47 | 0.065 |
| NUCLEAR_IMPORT | 46 | −1.47 | 0.065 |
| NEGATIVE_REGULATION_OF_SIGNAL_TRANSDUCTION | 34 | −1.47 | 0.065 |
| ESTABLISHMENT_OF_PROTEIN_LOCALIZATION | 173 | −1.47 | 0.066 |
| CELL_CELL_SIGNALING | 291 | −1.46 | 0.068 |
| CARBOXYLIC_ACID_TRANSPORT | 36 | −1.46 | 0.068 |
| FATTY_ACID_METABOLIC_PROCESS | 52 | −1.46 | 0.070 |
| POSITIVE_REGULATION_OF_BINDING | 23 | −1.46 | 0.071 |
| NEURON_DIFFERENTIATION | 58 | −1.45 | 0.072 |
| POSITIVE_REGULATION_OF_TRANSCRIPTION_FACTOR_ACTIVITY | 20 | −1.45 | 0.072 |
| ORGANIC_ACID_TRANSPORT | 37 | −1.45 | 0.074 |
| INTRACELLULAR_TRANSPORT | 263 | −1.44 | 0.076 |
| REGULATION_OF_ANGIOGENESIS | 17 | −1.44 | 0.077 |
| CELL_CYCLE_CHECKPOINT_GO_0000075 | 42 | −1.44 | 0.079 |
| RESPONSE_TO_HYPOXIA | 23 | −1.44 | 0.080 |
| CELL_CELL_ADHESION | 70 | −1.43 | 0.081 |
| MACROMOLECULE_LOCALIZATION | 216 | −1.43 | 0.082 |
| NUCLEOTIDE_BIOSYNTHETIC_PROCESS | 16 | −1.43 | 0.082 |
| INTERPHASE_OF_MITOTIC_CELL_CYCLE | 57 | −1.43 | 0.083 |
| G_PROTEIN_SIGNALING_COUPLED_TO_CYCLIC_NUCLEOTIDE_SECOND_MESSENGER | 68 | −1.42 | 0.087 |
| INDUCTION_OF_APOPTOSIS_BY_INTRACELLULAR_SIGNALS | 21 | −1.42 | 0.087 |
| REGULATION_OF_TRANSCRIPTION_FACTOR_ACTIVITY | 34 | −1.42 | 0.087 |
| REGULATION_OF_INTRACELLULAR_TRANSPORT | 23 | −1.42 | 0.087 |
| CELLULAR_LOCALIZATION | 340 | −1.41 | 0.090 |
| INTRACELLULAR_PROTEIN_TRANSPORT | 136 | −1.41 | 0.090 |
| AMINO_ACID_CATABOLIC_PROCESS | 19 | −1.41 | 0.092 |
| POSITIVE_REGULATION_OF_PHOSPHORYLATION | 16 | −1.41 | 0.093 |
| NEGATIVE_REGULATION_OF_CELL_CYCLE | 73 | −1.41 | 0.093 |
| ANATOMICAL_STRUCTURE_FORMATION | 42 | −1.40 | 0.095 |
| EPIDERMIS_DEVELOPMENT | 48 | −1.40 | 0.095 |
| ANATOMICAL_STRUCTURE_MORPHOGENESIS | 303 | −1.40 | 0.096 |
| TRANSMEMBRANE_RECEPTOR_PROTEIN_TYROSINE_KINASE_SIGNALING_PATHWAY | 70 | −1.40 | 0.098 |
| CELL_CYCLE_ARREST_GO_0007050 | 52 | −1.40 | 0.098 |
| GROWTH | 60 | −1.40 | 0.098 |
| ENDOSOME_TRANSPORT | 23 | −1.39 | 0.098 |
| REGULATION_OF_CELLULAR_COMPONENT_ORGANIZATION_AND_BIOGENESIS | 114 | −1.39 | 0.102 |
| REGULATION_OF_NUCLEOCYTOPLASMIC_TRANSPORT | 20 | −1.39 | 0.102 |
| ACTIVATION_OF_NF_KAPPAB_TRANSCRIPTION_FACTOR | 16 | −1.39 | 0.103 |
| JNK_CASCADE | 45 | −1.38 | 0.104 |
| ORGANELLE_ORGANIZATION_AND_BIOGENESIS | 431 | −1.38 | 0.104 |
| POSITIVE_REGULATION_OF_TRANSPORT | 18 | −1.38 | 0.105 |
| AMINE_CATABOLIC_PROCESS | 20 | −1.38 | 0.104 |
| AMINO_ACID_AND_DERIVATIVE_METABOLIC_PROCESS | 84 | −1.38 | 0.107 |
| CELL_PROJECTION_BIOGENESIS | 26 | −1.38 | 0.109 |
| NITROGEN_COMPOUND_CATABOLIC_PROCESS | 22 | −1.37 | 0.109 |
| ALCOHOL_METABOLIC_PROCESS | 73 | −1.37 | 0.110 |
| CELL_MATRIX_ADHESION | 33 | −1.37 | 0.109 |
| MACROMOLECULE_CATABOLIC_PROCESS | 120 | −1.37 | 0.109 |
| MONOVALENT_INORGANIC_CATION_TRANSPORT | 67 | −1.36 | 0.119 |
| ECTODERM_DEVELOPMENT | 56 | −1.35 | 0.125 |
| ENZYME_LINKED_RECEPTOR_PROTEIN_SIGNALING_PATHWAY | 119 | −1.35 | 0.125 |
| GENERATION_OF_NEURONS | 64 | −1.35 | 0.125 |
| PHOSPHOINOSITIDE_BIOSYNTHETIC_PROCESS | 24 | −1.35 | 0.127 |
| RESPONSE_TO_ORGANIC_SUBSTANCE | 27 | −1.35 | 0.127 |
| PROTEIN_AMINO_ACID_N_LINKED_GLYCOSYLATION | 27 | −1.34 | 0.129 |
| MUSCLE_DEVELOPMENT | 77 | −1.34 | 0.131 |
| LIPID_BIOSYNTHETIC_PROCESS | 84 | −1.34 | 0.131 |
| MITOCHONDRIAL_TRANSPORT | 20 | −1.34 | 0.133 |
| ER_NUCLEAR_SIGNALING_PATHWAY | 16 | −1.34 | 0.134 |
| PHOSPHOLIPID_METABOLIC_PROCESS | 66 | −1.34 | 0.134 |
| REGULATION_OF_GROWTH | 45 | −1.33 | 0.135 |
| ANION_TRANSPORT | 18 | −1.33 | 0.135 |
| SISTER_CHROMATID_SEGREGATION | 15 | −1.33 | 0.135 |
| COFACTOR_BIOSYNTHETIC_PROCESS | 21 | −1.33 | 0.136 |
| REGULATION_OF_CELL_DIFFERENTIATION | 47 | −1.33 | 0.138 |
| RHYTHMIC_PROCESS | 18 | −1.33 | 0.138 |
| CYCLIC_NUCLEOTIDE_MEDIATED_SIGNALING | 70 | −1.33 | 0.139 |
| REGULATION_OF_GENE_EXPRESSION | 595 | −1.32 | 0.140 |
| BONE_REMODELING | 23 | −1.32 | 0.140 |
| NEUROGENESIS | 74 | −1.32 | 0.140 |
| PROTEIN_TARGETING | 101 | −1.32 | 0.141 |
| RESPONSE_TO_OXIDATIVE_STRESS | 39 | −1.32 | 0.143 |
| CELL_SUBSTRATE_ADHESION | 34 | −1.32 | 0.144 |
| NUCLEOCYTOPLASMIC_TRANSPORT | 83 | −1.31 | 0.153 |
| CELLULAR_BIOSYNTHETIC_PROCESS | 281 | −1.30 | 0.157 |
| REGULATION_OF_TRANSPORT | 56 | −1.30 | 0.161 |
| PROTEIN_IMPORT | 57 | −1.29 | 0.166 |
| NUCLEAR_TRANSPORT | 84 | −1.29 | 0.170 |
| MEMBRANE_FUSION | 26 | −1.29 | 0.171 |
| REPRODUCTIVE_PROCESS | 102 | −1.28 | 0.173 |
| NEGATIVE_REGULATION_OF_CELLULAR_COMPONENT_ORGANIZATION_AND_BIOGENESIS | 26 | −1.28 | 0.173 |
| REGULATION_OF_NUCLEOBASE_NUCLEOSIDE_NUCLEOTIDE_AND_{—} | 541 | −1.27 | 0.182 |
| NUCLEIC_ACID_METABOLIC_PROCESS | | | |
| REGULATION_OF_TRANSCRIPTION | 503 | −1.27 | 0.184 |
| NUCLEAR_ORGANIZATION_AND_BIOGENESIS | 25 | −1.27 | 0.183 |
| GLYCEROPHOSPHOLIPID_METABOLIC_PROCESS | 43 | −1.27 | 0.183 |
| MICROTUBULE_BASED_PROCESS | 77 | −1.27 | 0.185 |
| NEURON_DEVELOPMENT | 50 | −1.27 | 0.185 |
| CELL_STRUCTURE_DISASSEMBLY_DURING_APOPTOSIS | 16 | −1.26 | 0.191 |
| TISSUE_REMODELING | 24 | −1.26 | 0.196 |
| SKELETAL_DEVELOPMENT | 78 | −1.26 | 0.197 |
| CELLULAR_COMPONENT_ASSEMBLY | 269 | −1.26 | 0.197 |
| NEGATIVE_REGULATION_OF_CELLULAR_PROTEIN_METABOLIC_PROCESS | 42 | −1.25 | 0.203 |
| REGULATION_OF_TRANSLATION | 80 | −1.25 | 0.203 |
| VITAMIN_METABOLIC_PROCESS | 15 | −1.25 | 0.205 |
| NEGATIVE_REGULATION_OF_PROTEIN_METABOLIC_PROCESS | 44 | −1.24 | 0.208 |
| MACROMOLECULAR_COMPLEX_ASSEMBLY | 256 | −1.24 | 0.209 |
| AMINO_SUGAR_METABOLIC_PROCESS | 18 | −1.24 | 0.209 |
| NUCLEOTIDE_METABOLIC_PROCESS | 35 | −1.23 | 0.218 |
| BIOSYNTHETIC_PROCESS | 417 | −1.23 | 0.218 |
| RESPONSE_TO_UV | 22 | −1.23 | 0.218 |
| ACTIVATION_OF_PROTEIN_KINASE_ACTIVITY | 24 | −1.23 | 0.222 |
| REGULATION_OF_MYELOID_CELL_DIFFERENTIATION | 17 | −1.23 | 0.222 |
| ACTIN_POLYMERIZATION_AND_OR_DEPOLYMERIZATION | 20 | −1.22 | 0.227 |
| TRANSCRIPTION_DNA_DEPENDENT | 564 | −1.22 | 0.229 |
| TRANSCRIPTION_FROM_RNA_POLYMERASE_II_PROMOTER | 410 | −1.22 | 0.229 |
| REGULATION_OF_TRANSCRIPTION_DNA_DEPENDENT | 411 | −1.22 | 0.236 |
| RNA_CATABOLIC_PROCESS | 20 | −1.21 | 0.241 |
| TRANSFORMING_GROWTH_FACTOR_BETA_RECEPTOR_SIGNALING_PATHWAY | 31 | −1.21 | 0.242 |
| RNA_BIOSYNTHETIC_PROCESS | 566 | −1.21 | 0.241 |
| GLYCOPROTEIN_METABOLIC_PROCESS | 84 | −1.21 | 0.241 |
| FEMALE_PREGNANCY | 26 | −1.21 | 0.246 |
| REGULATION_OF_RNA_METABOLIC_PROCESS | 414 | −1.20 | 0.250 |
| SECRETION_BY_CELL | 99 | −1.20 | 0.249 |
| PHOSPHOINOSITIDE_METABOLIC_PROCESS | 30 | −1.20 | 0.250 |
| REGULATION_OF_CYTOSKELETON_ORGANIZATION_AND_BIOGENESIS | 29 | −1.20 | 0.253 |
| NEGATIVE_REGULATION_OF_METABOLIC_PROCESS | 238 | −1.20 | 0.254 |
| NEGATIVE_REGULATION_OF_CELLULAR_METABOLIC_PROCESS | 237 | −1.19 | 0.257 |
| REGULATION_OF_MITOTIC_CELL_CYCLE | 19 | −1.18 | 0.270 |
| STEROID_BIOSYNTHETIC_PROCESS | 17 | −1.18 | 0.282 |
| PROTEOGLYCAN_METABOLIC_PROCESS | 16 | −1.18 | 0.282 |
| NEGATIVE_REGULATION_OF_TRANSCRIPTION_FROM_RNA_POLYMERASE_II_PROMOTER | 80 | −1.17 | 0.282 |
| POSITIVE_REGULATION_OF_TRANSCRIPTION | 127 | −1.17 | 0.283 |
| NEGATIVE_REGULATION_OF_TRANSCRIPTION_DNA_DEPENDENT | 121 | −1.16 | 0.305 |
| RESPONSE_TO_NUTRIENT_LEVELS | 19 | −1.15 | 0.313 |
| NEGATIVE_REGULATION_OF_TRANSCRIPTION | 177 | −1.15 | 0.315 |
| POSITIVE_REGULATION_OF_NUCLEOBASE_NUCLEOSIDE_NUCLEOTIDE_AND_NUCLEIC_{—} | 133 | −1.15 | 0.318 |
| ACID_METABOLIC_PROCESS | | | |
| NEGATIVE_REGULATION_OF_RNA_METABOLIC_PROCESS | 121 | −1.15 | 0.321 |
| GLYCEROPHOSPHOLIPID_BIOSYNTHETIC_PROCESS | 30 | −1.14 | 0.324 |
| ION_TRANSPORT | 129 | −1.14 | 0.326 |
| DETECTION_OF_STIMULUS | 26 | −1.14 | 0.326 |
| PROTEIN_PROCESSING | 43 | −1.14 | 0.326 |
| REGULATION_OF_TRANSCRIPTION_FROM_RNA_POLYMERASE_II_PROMOTER | 261 | −1.14 | 0.328 |
| BIOPOLYMER_CATABOLIC_PROCESS | 107 | −1.14 | 0.330 |
| NEGATIVE_REGULATION_OF_NUCLEOBASE_NUCLEOSIDE_NUCLEOTIDE_AND_NUCLEIC_{—} | 193 | −1.13 | 0.339 |
| ACID_METABOLIC_PROCESS | | | |
| COENZYME_METABOLIC_PROCESS | 35 | −1.13 | 0.342 |
| SULFUR_METABOLIC_PROCESS | 34 | −1.13 | 0.346 |
| EXOCYTOSIS | 21 | −1.13 | 0.347 |
| NEGATIVE_REGULATION_OF_TRANSPORT | 18 | −1.12 | 0.356 |
| PROTEIN_AUTOPROCESSING | 29 | −1.12 | 0.356 |
| INSULIN_RECEPTOR_SIGNALING_PATHWAY | 19 | −1.12 | 0.358 |
| RESPONSE_TO_ABIOTIC_STIMULUS | 73 | −1.12 | 0.360 |
| PROTEIN_AMINO_ACID_AUTOPHOSPHORYLATION | 29 | −1.11 | 0.362 |
| REGULATION_OF_GENE_EXPRESSION_EPIGENETIC | 28 | −1.11 | 0.368 |
| PHOSPHOLIPID_BIOSYNTHETIC_PROCESS | 38 | −1.11 | 0.369 |
| NEGATIVE_REGULATION_OF_MAP_KINASE_ACTIVITY | 16 | −1.11 | 0.372 |
| REGUL^{N}_OF_CYCLIN_DEPENDENT_PROTEIN_KINASE_ACTIVITY | 39 | −1.10 | 0.392 |
| NITROGEN_COMPOUND_BIOSYNTHETIC_PROCESS | 23 | −1.09 | 0.407 |
| RESPONSE_TO_HORMONE_STIMULUS | 27 | −1.09 | 0.407 |
| CALCIUM_MEDIATED_SIGNALING | 15 | −1.08 | 0.424 |
| REGULATION_OF_SECRETION | 30 | −1.07 | 0.433 |
| TRANSMEMBRANE_RECEPTOR_PROTEIN_SERINE_THREONINE_KINASE_SIGNALING_PATHWAY | 41 | −1.06 | 0.450 |
| AROMATIC_COMPOUND_METABOLIC_PROCESS | 25 | −1.06 | 0.454 |
| NEGATIVE_REGULATION_OF_CELL_DIFFERENTIATION | 21 | −1.06 | 0.454 |
| DNA_DAMAGE_RESPONSE_SIGNAL_TRANSDUCTION | 31 | −1.06 | 0.458 |
| GLUCOSE_METABOLIC_PROCESS | 23 | −1.06 | 0.457 |
| REGULATION_OF_CYTOKINE_PRODUCTION | 20 | −1.06 | 0.456 |
| PROTEIN_HOMOOLIGOMERIZATION | 20 | −1.05 | 0.462 |
| REPRODUCTION | 172 | −1.05 | 0.464 |
| NUCLEOBASE_NUCLEOSIDE_AND_NUCLEOTIDE_METABOLIC_PROCESS | 45 | −1.05 | 0.463 |
| PHOSPHOINOSITIDE_MEDIATED_SIGNALING | 36 | −1.04 | 0.476 |
| ESTABLISHMENT_AND_OR_MAINTENANCE_OF_CELL_POLARITY | 18 | −1.04 | 0.484 |
| APOPTOTIC_NUCLEAR_CHANGES | 17 | −1.03 | 0.496 |
| CARBOHYDRATE_TRANSPORT | 16 | −1.02 | 0.509 |
| EPIDERMAL_GROWTH_FACTOR_RECEPTOR_SIGNALING_PATHWAY | 18 | −1.02 | 0.511 |
| SYSTEM_PROCESS | 393 | −1.02 | 0.512 |
| CELLULAR_COMPONENT_DISASSEMBLY | 30 | −1.02 | 0.524 |
| MICROTUBULE_CYTOSKELETON_ORGANIZ^{N}_AND_BIOGENESIS | 33 | −1.01 | 0.528 |
| PROTEIN_POLYMERIZATION | 16 | −1.01 | 0.534 |
| MEMBRANE_LIPID_BIOSYNTHETIC_PROCESS | 44 | −1.01 | 0.533 |
| CATION_TRANSPORT | 107 | −1.01 | 0.532 |
| SULFUR_COMPOUND_BIOSYNTHETIC_PROCESS | 15 | −1.00 | 0.542 |
| G_PROTEIN_SIGNALING_COUPLED_TO_CAMP_NUCLEOTIDE_SECOND_MESSENGER | 44 | −1.00 | 0.556 |
| NEURITE_DEVELOPMENT | 43 | −1.00 | 0.555 |
| TRANSCRIPT^{N}_INITIATION_FROM_RNA_POLYMERASEII_PROMOTER | 28 | −0.99 | 0.555 |
| NERVOUS_SYSTEM_DEVELOPMENT | 291 | −0.99 | 0.556 |
| POSITIVE_REGULATION_OF_TRANSCRIPTION_DNA_DEPENDENT | 106 | −0.99 | 0.555 |
| INTRACELLULAR_RECEPTOR_MEDIATED_SIGNALING_PATHWAY | 20 | −0.99 | 0.554 |
| CAMP_MEDIATED_SIGNALING | 45 | −0.99 | 0.555 |
| RESPONSE_TO_RADIATION | 47 | −0.99 | 0.556 |
| REGULATION_OF_MAPKKK_CASCADE | 17 | −0.99 | 0.557 |
| POSITIVE_REGULATION_OF_RNA_METABOLIC_PROCESS | 107 | −0.99 | 0.560 |
| NEGATIVE_REGULATION_OF_BINDING | 16 | −0.98 | 0.574 |
| AXONOGENESIS | 36 | −0.98 | 0.579 |
| STRIATED_MUSCLE_DEVELOPMENT | 31 | −0.98 | 0.578 |
| NEGATIVE_REGULATION_OF_DNA_BINDING | 15 | −0.98 | 0.580 |
| SECRETION | 143 | −0.97 | 0.583 |
| TRNA_METABOLIC_PROCESS | 19 | −0.97 | 0.596 |
| NEGATIVE_REGULATION_OF_TRANSLATION | 20 | −0.96 | 0.598 |
| DNA_INTEGRITY_CHECKPOINT | 21 | −0.95 | 0.626 |
| REGULATION_OF_ORGANELLE_ORGANIZATION_AND_BIOGENESIS | 38 | −0.95 | 0.635 |
| CYTOSKELETON_DEPENDENT_INTRACELLULAR_TRANSPORT | 23 | −0.95 | 0.633 |
| G_PROTEIN_SIGNALING_COUPLED_TO_IP3_SECOND_MESSENGER_PHOSPHOLIPASE_C_ACTIVATING | 33 | −0.94 | 0.643 |
| HEART_DEVELOPMENT | 29 | −0.93 | 0.653 |
| POTASSIUM_ION_TRANSPORT | 38 | −0.93 | 0.660 |
| SYNAPTOGENESIS | 15 | −0.93 | 0.666 |
| RESPONSE_TO_LIGHT_STIMULUS | 34 | −0.92 | 0.673 |
| DNA_CATABOLIC_PROCESS | 20 | −0.91 | 0.685 |
| NEGATIVE_REGULATION_OF_CELLULAR_BIOSYNTHETIC_PROCESS | 26 | −0.91 | 0.685 |
| TRANSLATION | 159 | −0.91 | 0.686 |
| SENSORY_PERCEPTION | 132 | −0.91 | 0.691 |
| EXTRACELLULAR_STRUCTURE_ORGANIZATION_AND_BIOGENESIS | 26 | −0.90 | 0.700 |
| SECRETORY_PATHWAY | 75 | −0.90 | 0.714 |
| CELLULAR_PROTEIN_CATABOLIC_PROCESS | 56 | −0.89 | 0.717 |
| NEGATIVE_REGULATION_OF_BIOSYNTHETIC_PROCESS | 27 | −0.89 | 0.724 |
| POLYSACCHARIDE_METABOLIC_PROCESS | 16 | −0.89 | 0.727 |
| OXYGEN_AND_REACTIVE_OXYGEN_SPECIES_METABOLIC_PROCESS | 19 | −0.88 | 0.728 |
| REGULATION_OF_DNA_METABOLIC_PROCESS | 34 | −0.88 | 0.736 |
| ONE_CARBON_COMPOUND_METABOLIC_PROCESS | 25 | −0.87 | 0.748 |
| REGULATION_OF_NEUROTRANSMITTER_LEVELS | 17 | −0.87 | 0.750 |
| MACROMOLECULE_BIOSYNTHETIC_PROCESS | 289 | −0.87 | 0.751 |
| DNA_DAMAGE_CHECKPOINT | 18 | −0.86 | 0.764 |
| PROTEIN_CATABOLIC_PROCESS | 65 | −0.86 | 0.772 |
| CELLULAR_MORPHOGENESIS_DURING_DIFFERENTIATION | 42 | −0.85 | 0.772 |
| TRANSCRIPTION_INITIATION | 34 | −0.85 | 0.779 |
| NEURON_APOPTOSIS | 15 | −0.85 | 0.780 |
| NEUROLOGICAL_SYSTEM_PROCESS | 261 | −0.85 | 0.780 |
| ELECTRON_TRANSPORT_GO_0006118 | 47 | −0.85 | 0.779 |
| STEROID_HORMONE_RECEPTOR_SIGNALING_PATHWAY | 19 | −0.83 | 0.810 |
| DNA_METABOLIC_PROCESS | 227 | −0.82 | 0.816 |
| SKELETAL_MUSCLE_DEVELOPMENT | 23 | −0.81 | 0.827 |
| GOLGI_VESICLE_TRANSPORT | 46 | −0.81 | 0.831 |
| SYNAPTIC_TRANSMISSION | 115 | −0.81 | 0.835 |
| LIPOPROTEIN_METABOLIC_PROCESS | 31 | −0.80 | 0.844 |
| NUCLEAR_EXPORT | 34 | −0.80 | 0.845 |
| METAL_ION_TRANSPORT | 85 | −0.79 | 0.854 |
| GLYCOPROTEIN_BIOSYNTHETIC_PROCESS | 68 | −0.79 | 0.852 |
| SYNAPSE_ORGANIZATION_AND_BIOGENESIS | 19 | −0.79 | 0.852 |
| MORPHOGENESIS_OF_AN_EPITHELIUM | 15 | −0.78 | 0.858 |
| TRANSMISSION_OF_NERVE_IMPULSE | 129 | −0.78 | 0.867 |
| PROTEIN_UBIQUITINATION | 37 | −0.75 | 0.899 |
| PROTEIN_FOLDING | 50 | −0.74 | 0.904 |
| DIGESTION | 22 | −0.74 | 0.906 |
| BRAIN_DEVELOPMENT | 41 | −0.72 | 0.930 |
| SODIUM_ION_TRANSPORT | 16 | −0.69 | 0.952 |
| DNA_PACKAGING | 30 | −0.68 | 0.954 |
| ADENYLATE_CYCLASE_ACTIVATION | 16 | −0.68 | 0.952 |
| CARBOHYDRATE_BIOSYNTHETIC_PROCESS | 43 | −0.66 | 0.961 |
| G_PROTEIN_SIGNALING_ADENYLATE_CYCLASE_ACTIVATING_PATHWAY | 19 | −0.65 | 0.967 |
| UBIQUITIN_CYCLE | 44 | −0.64 | 0.970 |
| PROTEIN_MODIFICATION_BY_SMALL_PROTEIN_CONJUGATION | 40 | −0.64 | 0.970 |
| RESPONSE_TO_ENDOGENOUS_STIMULUS | 176 | −0.64 | 0.968 |
| ER_TO_GOLGI_VESICLE_MEDIATED_TRANSPORT | 18 | −0.62 | 0.974 |
| GENERAT^{N}_OF_A_SIGNAL_INVOLVED_IN_CELL_CELL_SIGNALING | 21 | −0.59 | 0.982 |
| MICROTUBULE_BASED_MOVEMENT | 15 | −0.43 | 0.999 |

TABLE 5

Signal transduction
CYTOKINE AND
CHEMOKINE MEDIATED
SIGNALING PATHWAY
CELL SURFACE RECEPTOR
LINKED SIGNAL
TRANSDUCTION GO0007166
SMALL GTPASE MEDIATED
SIGNAL TRANSDUCTION
RAS PROTEIN SIGNAL
TRANSDUCTION
RHO PROTEIN SIGNAL
TRANSDUCTION
PROTEIN KINASE CASCADE
INTRACELLULAR
SIGNALING CASCADE
REGULATION OF SIGNAL
TRANSDUCTION
POSITIVE REGULATION OF
SIGNAL TRANSDUCTION
I KAPPAB KINASE NF
KAPPAB CASCADE
REGULATION OF I KAPPAB
KINASE NF KAPPAB
CASCADE
POSITIVE REGULATION OF I
KAPPAB KINASE NF KAPPAB
CASCADE
JAK STAT CASCADE
REGULATION OF PROTEIN
KINASE ACTIVITY
REGULATION OF KINASE
ACTIVITY
REGULATION OF
TRANSFERASE ACTIVITY
POSITIVE REGULATION OF
TRANSFERASE ACTIVITY
ACTIVATION OF MAPK
ACTIVITY
REGULATION OF
CATALYTIC ACTIVITY
POSITIVE REGULATION OF
CATALYTIC ACTIVITY
REGULATION OF JNK
ACTIVITY
POSITIVE REGULATION OF
JNK ACTIVITY
ACTIVATION OF JNK
ACTIVITY
PEPTIDYL TYROSINE
MODIFICATION
PEPTIDYL TYROSINE
PHOSPHORYLATION
PROTEIN AMINO ACID
PHOSPHORYLATION
PHOSPHORYLATION
PROTEIN AMINO ACID
DEPHOSPHORYLATION
DEPHOSPHORYLATION
Cytokine production
CYTOKINE PRODUCTION
CYTOKINE METABOLIC
PROCESS
CYTOKINE BIOSYNTHETIC
PROCESS
REGULATION OF CYTOKINE
BIOSYNTHETIC PROCESS
Programmed cell death
APOPTOTIC PROGRAM
REGULATION OF
HYDROLASE ACTIVITY
POSITIVE REGULATION OF
HYDROLASE ACTIVITY
POSITIVE REGULATION OF
CASPASE ACTIVITY
CASPASE ACTIVATION
NEGATIVE REGULATION OF
DEVELOPMENTAL PROCESS
NEGATIVE REGULATION OF
APOPTOSIS
NEGATIVE REGULATION OF
PROGRAMMED CELL DEATH
ANTI APOPTOSIS
REGULATION OF
DEVELOPMENTAL PROCESS
REGULATION OF APOPTOSIS
REGULATION OF
PROGRAMMED CELL DEATH
APOPTOSIS
PROGRAMMED CELL DEATH
CELL DEVELOPMENT
POSITIVE REGULATION OF
DEVELOPMENTAL PROCESS
Mitosis
CYTOKINESIS
G1 S TRANSITION OF
MITOTIC CELL CYCLE
MITOTIC CELL CYCLE
M PHASE OF MITOTIC CELL
CYCLE
MITOSIS
REGULATION OF MITOSIS
Leukocyte activation
CELL ACTIVATION
LEUKOCYTE ACTIVATION
LYMPHOCYTE ACTIVATION
T CELL ACTIVATION
B CELL ACTIVATION
REGULATION OF
LYMPHOCYTE ACTIVATION
POSITIVE REGULATION OF
LYMPHOCYTE ACTIVATION
REGULATION OF T CELL
ACTIVATION
T CELL PROLIFERATION
LEUKOCYTE
DIFFERENTIATION
LYMPHOCYTE
DIFFERENTIATION
Homeostatic process
HOMEOSTASIS OF NUMBER
OF CELLS
REGULATION OF
BIOLOGICAL QUALITY
HOMEOSTATIC PROCESS
CHEMICAL HOMEOSTASIS
ION HOMEOSTASIS
CATION HOMEOSTASIS
CELLULAR CATION
HOMEOSTASIS
CELLULAR HOMEOSTASIS
Immune system development
HEMOPOIESIS
IMMUNE SYSTEM
DEVELOPMENT
HEMOPOIETIC OR
LYMPHOID ORGAN
DEVELOPMENT
MYELOID CELL
DIFFERENTIATION
Other
PROTEOLYSIS
CELLULAR LIPID
CATABOLIC PROCESS
LIPID CATABOLIC PROCESS
ORGANIC ACID METABOLIC
PROCESS
CARBOXYLIC ACID
METABOLIC PROCESS
MEMBRANE
ORGANIZATION AND
BIOGENESIS
RECEPTOR MEDIATED
ENDOCYTOSIS
PIGMENT BIOSYNTHETIC
PROCESS
PROTEIN COMPLEX
ASSEMBLY
REGULATION OF CELL
ADHESION
VIRAL REPRODUCTIVE
PROCESS
LIPID TRANSPORT
REGULATION OF
ANATOMICAL STRUCTURE
MORPHOGENESIS
REGULATION OF CELL
MORPHOGENESIS
ACTIN CYTOSKELETON
ORGANIZATION AND
BIOGENESIS
ACTIN FILAMENT BASED
PROCESS
POSITIVE REGULATION OF
TRANSLATION

TABLE 6

| Name | Symbol |
|---|---|
| MAD2 mitotic arrest deficient-like 2 (yeast) | MAD2L2 |
| olfactory receptor, family 2, subfamily A, member 7 | OR2A7 |
| tubulin tyrosine ligase-like family, member 7 | TTLL7 |
| G protein-coupled receptor 68 | GPR68 |
| T-cell, immune regulator 1, ATPase, H+ transporting, lysosomal V0 | TCIRG1 |
| subunit A3 | |
| PYD and CARD domain containing | PYCARD |
| SRY (sex determining region Y)-box 5 | SOX5 |
| thymidine phosphorylase /// outer dense fiber of sperm tails 3B | TYMP /// ODF3B |
| ribonuclease, RNase A family, k6 | RNASE6 |
| Wiskott-Aldrich syndrome (eczema-thrombocytopenia) | WAS |
| proline rich 11 | PRR11 |
| acyl-CoA thioesterase 7 /// acyl-CoA thioesterase 7 pseudogene | ACOT7 /// |
| | LOC344967 |
| coactosin-like 1 (Dictyostelium) | COTL1 |
| antigen identified by monoclonal antibody Ki-67 | MKI67 |
| mannosyl (alpha-1,3-)-glycoprotein beta-1,2-N- | MGAT1 |
| acetylglucosaminyltransferase | |
| tumor necrosis factor, alpha-induced protein 2 | TNFAIP2 |
| hypothetical LOC349196 | LOC349196 |
| tweety homolog 3 (Drosophila) | TTYH3 |
| cerebral cavernous malformation 2 | CCM2 |
| serpin peptidase inhibitor, clade E (nexin, plasminogen activator inhibitor | SERPINE1 |
| type 1), member 1 | |
| adenosine monophosphate deaminase 2 (isoform L) | AMPD2 |
| mannosidase, alpha, class 2B, member 1 | MAN2B1 |
| suppressor of cytokine signaling 3 | SOCS3 |
| G protein-coupled receptor kinase 6 | GRK6 |
| c-src tyrosine kinase | CSK |
| heme oxygenase (decycling) 1 | HMOX1 |
| histone cluster 1, H3b | HIST1H3B |
| T-cell activation RhoGTPase activating protein | TAGAP |
| colony stimulating factor 1 receptor | CSF1R |
| rhomboid 5 homolog 2 (Drosophila) | RHBDF2 |
| minichromosome maintenance complex component 5 | MCM5 |
| TBC1 domain family, member 9 (with GRAM domain) | TBC1D9 |
| chromosome 6 open reading frame 150 | C6orf150 |
| sulfiredoxin 1 homolog (S. cerevisiae) | SRXN1 |
| chromosome 17 open reading frame 62 | C17orf62 |
| family with sequence similarity 111, member B | FAM111B |
| actin related protein 2/3 complex, subunit 1B, 41 kDa /// actin related | ARPC1B /// |
| protein 2/3 complex, subunit 1A, 41 kDa | ARPC1A |
| FYVE, RhoGEF and PH domain containing 2 | FGD2 |
| polo-like kinase 3 (Drosophila) | PLK3 |
| chromosome 10 open reading frame 55 /// plasminogen activator, | C10orf55 /// PLAU |
| urokinase | |
| cysteinyl leukotriene receptor 2 | CYSLTR2 |
| intercellular adhesion molecule 3 | ICAM3 |
| signal transducer and activator of transcription 5A | STAT5A |
| protein kinase, AMP-activated, beta 2 non-catalytic subunit | PRKAB2 |
| solute carrier family 15, member 3 | SLC15A3 |
| platelet-activating factor receptor | PTAFR |
| glucose-6-phosphate dehydrogenase | G6PD |
| hematopoietic cell-specific Lyn substrate 1 | HCLS1 |
| lymphocyte antigen 96 | LY96 |
| zinc finger, DHHC-type containing 18 | ZDHHC18 |
| zinc finger protein 507 | ZNF507 |
| hematological and neurological expressed 1 | HN1 |
| acid phosphatase 2, lysosomal | ACP2 |
| spleen focus forming virus (SFFV) proviral integration oncogene spi1 | SPI1 |
| ethylmalonic encephalopathy 1 | ETHE1 |
| CKLF-like MARVEL transmembrane domain containing 7 | CMTM7 |
| family with sequence similarity 26, member F | FAM26F |
| KIAA1539 | KIAA1539 |
| chromosome 1 open reading frame 152 /// phosphodiesterase 4D | C1orf152 /// |
| interacting protein | PDE4DIP |
| melanoma antigen family A, 5 | MAGEA5 |
| aspartate beta-hydroxylase domain containing 2 | ASPHD2 |
| paired immunoglobin-like type 2 receptor alpha | PILRA |
| potassium voltage-gated channel, shaker-related subfamily, beta member 2 | KCNAB2 |
| interferon induced transmembrane protein 1 (9-27) | IFITM1 |
| chromosome 7 open reading frame 41 | C7orf41 |
| interferon regulatory factor 8 | IRF8 |
| cathepsin Z | CTSZ |
| NFAT activating protein with ITAM motif 1 | NFAM1 |
| neutrophil cytosolic factor 4, 40 kDa | NCF4 |
| kelch-like 23 (Drosophila) /// phosphatase, orphan 2 | KLHL23 /// |
| | PHOSPHO2 |
| tumor necrosis factor (TNF superfamily, member 2) | TNF |
| ras homolog gene family, member G (rho G) | RHOG |
| interleukin 12 receptor, beta 1 | IL12RB1 |
| phosphatidylinositol 4-kinase type 2 alpha | PI4K2A |
| lectin, galactoside-binding, soluble, 9 | LGALS9 |
| matrix metallopeptidase 25 | MMP25 |
| EGFR-coamplified and overexpressed protein | ECOP |
| pim-1 oncogene | PIM1 |
| sorting nexin 20 | SNX20 |
| alcohol dehydrogenase, iron containing, 1 | ADHFE1 |
| formin-like 1 | FMNL1 |
| chemokine (C—X—C motif) ligand 2 | CXCL2 |
| lymphocyte-specific protein 1 | LSP1 |
| major histocompatibility complex, class II, DR beta 9 (pseudogene) | HLA-DRB9 |
| G protein-coupled receptor 18 | GPR18 |
| chemokine (C motif) ligand 1 | XCL1 |
| E2F transcription factor 3 | E2F3 |
| transporter 1, ATP-binding cassette, sub-family B (MDR/TAP) | TAP1 |
| secreted and transmembrane 1 | SECTM1 |
| myosin IF | MYO1F |
| lymphocyte-specific protein 1 pseudogene /// lymphocyte-specific protein | LOC654342 /// |
| 1 pseudogene /// lymphocyte-specific protein 1 | LOC645166 /// |
| | LSP1 |
| hypothetical protein LOC100127980 | LOC100127980 |
| protein tyrosine phosphatase, non-receptor type 6 | PTPN6 |
| hypothetical LOC440993 | LOC440993 |
| docking protein 1, 62 kDa (downstream of tyrosine kinase 1) | DOK1 |
| DKFZp761E198 protein | DKFZp761E198 |
| 2′-5′-oligoadenylate synthetase 3, 100 kDa | OAS3 |
| Abelson helper integration site 1 | AHI1 |
| methylenetetrahydrofolate dehydrogenase (NADP+ dependent) 2, | MTHFD2 |
| methenyltetrahydrofolate cyclohydrolase | |
| SLAM family member 8 /// chromosome 1 open reading frame 204 | SLAMF8 /// |
| | C1orf204 |
| Janus kinase 3 | JAK3 |
| solute carrier family 2 (facilitated glucose transporter), member 6 | SLC2A6 |
| dpy-19-like 2 pseudogene 1 (C. elegans) | DPY19L2P1 |
| alanyl (membrane) aminopeptidase | ANPEP |
| interleukin 1 receptor, type II | IL1R2 |
| potassium channel tetramerisation domain containing 5 | KCTD5 |
| CDC42 binding protein kinase alpha (DMPK-like) | CDC42BPA |
| early growth response 2 | EGR2 |
| complement component 5a receptor 1 | C5AR1 |
| transmembrane protein 149 | TMEM149 |
| phosphoinositide-3-kinase, catalytic, delta polypeptide | PIK3CD |
| G protein-coupled receptor 132 | GPR132 |
| phospholipase D family, member 3 | PLD3 |
| fermitin family homolog 3 (Drosophila) | FERMT3 |
| FYN binding protein (FYB-120/130) | FYB |
| neutrophil cytosolic factor 1 /// similar to Neutrophil cytosol factor 1 | NCF1 |
| (NCF-1) (Neutrophil NADPH oxidase factor 1) | |
| phospholipase B domain containing 2 | PLBD2 |
| chemokine (C—X—C motif) ligand 16 | CXCL16 |
| myeloid differentiation primary response gene (88) | MYD88 |
| serine/threonine kinase 17b | STK17B |
| nuclear factor (erythroid-derived 2)-like 3 | NFE2L3 |
| spleen tyrosine kinase | SYK |
| akirin 2 | AKIRIN2 |
| colony stimulating factor 2 receptor, beta, low-affinity (granulocyte- | CSF2RB |
| macrophage) | |
| cubilin (intrinsic factor-cobalamin receptor) | CUBN |
| histone cluster 1, H1b | HIST1H1B |
| formyl peptide receptor 3 | FPR3 |
| interleukin 21 receptor | IL21R |
| purinergic receptor P2Y, G-protein coupled, 13 | P2RY13 |
| WD repeat domain 76 | WDR76 |
| asp (abnormal spindle) homolog, microcephaly associated (Drosophila) | ASPM |
| bone morphogenetic protein receptor, type IA | BMPR1A |
| SH3 domain and tetratricopeptide repeats 1 | SH3TC1 |
| twinfilin, actin-binding protein, homolog 2 (Drosophila) /// toll-like | TWF2 /// TLR9 |
| receptor 9 | |
| egf-like module containing, mucin-like, hormone receptor-like 2 | EMR2 |
| InaD-like (Drosophila) | INADL |
| RELT tumor necrosis factor receptor | RELT |
| integrin, alpha X (complement component 3 receptor 4 subunit) | ITGAX |
| multiple PDZ domain protein | MPDZ |
| macrophage expressed 1 | MPEG1 |
| zinc finger, DBF-type containing 2 | ZDBF2 |
| PX19 protein pseudogene | LOC388955 |
| replication factor C (activator 1) 1, 145 kDa | RFC1 |
| cofilin 1 (non-muscle) | CFL1 |
| WD repeat domain 27 | WDR27 |
| PTK2B protein tyrosine kinase 2 beta | PTK2B |
| coronin, actin binding protein, 1A | CORO1A |
| interleukin 17 receptor A | IL17RA |
| NTF2-like export factor 1 | NXT1 |
| interferon gamma receptor 2 (interferon gamma transducer 1) | IFNGR2 |
| CTAGE family, member 4 | CTAGE4 |
| integrin, alpha L (antigen CD11A (p180), lymphocyte function-associated | ITGAL |
| antigen 1; alpha polypeptide) | |
| transporter 2, ATP-binding cassette, sub-family B (MDR/TAP) | TAP2 |
| G protein-coupled receptor 137B | GPR137B |
| C-type lectin domain family 10, member A | CLEC10A |
| G protein-coupled receptor 125 | GPR125 |
| hemopoietic cell kinase | HCK |
| S100 calcium binding protein A9 | S100A9 |
| family with sequence similarity 86, member A pseudogene | hCG_1990547 |
| integrin, beta 2 (complement component 3 receptor 3 and 4 subunit) | ITGB2 |
| GEM interacting protein | GMIP |
| NLR family, pyrin domain containing 3 | NLRP3 |
| myeloid cell leukemia sequence 1 (BCL2-related) | MCL1 |
| transcobalamin II; macrocytic anemia | TCN2 |
| Rho family GTPase 1 | RND1 |
| colony stimulating factor 2 receptor, alpha, low-affinity (granulocyte- | CSF2RA |
| macrophage) | |
| destrin (actin depolymerizing factor) | DSTN |
| olfactory receptor, family 7, subfamily E, member 13 pseudogene | OR7E13P |
| spectrin repeat containing, nuclear envelope 1 | SYNE1 |
| interleukin 10 receptor, alpha | IL10RA |
| WDFY family member 4 | WDFY4 |
| integrin, beta 7 | ITGB7 |
| CD6 molecule | CD6 |
| thyroid hormone receptor, beta (erythroblastic leukemia viral (v-erb-a) | THRB |
| oncogene homolog 2, avian) | |
| cyclin E1 | CCNE1 |
| ribonuclease T2 /// hypothetical protein LOC100131869 | RNASET2 /// |
| | LOC100131869 |
| phosphoinositide-3-kinase adaptor protein 1 | PIK3AP1 |
| DNA cross-link repair 1B (PSO2 homolog, S. cerevisiae) | DCLRE1B |
| Prader-Willi/Angelman syndrome-5 | PAR5 |
| ArfGAP with RhoGAP domain, ankyrin repeat and PH domain 1 | ARAP1 |
| glucuronidase, beta | GUSB |
| hypothetical protein LOC100132014 /// laeverin | LOC100132014 /// |
| | LVRN |
| Vac14 homolog (S. cerevisiae) | VAC14 |
| transketolase | TKT |
| interferon regulatory factor 1 | IRF1 |
| cathepsin A | CTSA |
| lysosomal-associated membrane protein 1 | LAMP1 |
| immediate early response 5 | IER5 |
| furry homolog (Drosophila) | FRY |
| family with sequence similarity 72, member D | FAM72D |
| ATP-binding cassette, sub-family D (ALD), member 1 | ABCD1 |
| hepatitis A virus cellular receptor 2 | HAVCR2 |
| tumor necrosis factor receptor superfamily, member 10b | TNFRSF10B |
| aminolevulinate, delta-, synthase 1 | ALAS1 |
| chromosome 1 open reading frame 38 | C1orf38 |
| LIM domain kinase 1 | LIMK1 |
| interleukin 1, beta | IL1B |
| regulator of G-protein signaling 18 | RGS18 |
| solute carrier family 26, member 5 (prestin) | SLC26A5 |
| sialic acid binding Ig-like lectin 7 | SIGLEC7 |
| major histocompatibility complex, class II, DQ beta 1 | HLA-DQB1 |
| Sec61 alpha 2 subunit (S. cerevisiae) | SEC61A2 |
| regulator of G-protein signaling 1 | RGS1 |
| chromodomain helicase DNA binding protein 6 | CHD6 |
| adaptor-related protein complex 2, sigma 1 subunit | AP2S1 |
| hypothetical LOC642980 | LOC642980 |
| SH3KBP1 binding protein 1 | SHKBP1 |
| CD5 molecule | CD5 |
| chromosome 17 open reading frame 87 | C17orf87 |
| carboxypeptidase, vitellogenic-like | CPVL |
| C-type lectin domain family 6, member A | CLEC6A |
| proline-serine-threonine phosphatase interacting protein 2 | PSTPIP2 |
| chromosome 6 open reading frame 124 | C6orf124 |
| ADP-ribosylation factor-like 11 | ARL11 |
| transmembrane protein 224 | TMEM224 |
| C-type lectin domain family 7, member A | CLEC7A |
| solute carrier family 24 (sodium/potassium/calcium exchanger), member 6 | SLC24A6 |
| adenosine deaminase | ADA |
| NAD kinase | NADK |
| transmembrane protein 206 | TMEM206 |
| PRELI domain containing 1 /// PX19 protein pseudogene | PRELID1 /// |
| | LOC388955 |
| interleukin 27 receptor, alpha | IL27RA |
| basic helix-loop-helix family, member e40 | BHLHE40 |
| Fas apoptotic inhibitory molecule 3 | FAIM3 |
| immunoglobulin superfamily, member 6 | IGSF6 |
| ribosomal protein S6 kinase, 90 kDa, polypeptide 4 | RPS6KA4 |
| interleukin 8 receptor, beta | IL8RB |
| topoisomerase (DNA) II alpha 170 kDa | TOP2A |
| CMT1A duplicated region transcript 4 /// family with sequence similarity | CDRT4 /// |
| 18, member B2 | FAM18B2 |
| solute carrier family 16, member 6 (monocarboxylic acid transporter 7) | SLC16A6 |
| dipeptidyl-peptidase 9 | DPP9 |
| serine hydroxymethyltransferase 2 (mitochondrial) | SHMT2 |
| C-type lectin domain family 9, member A | CLEC9A |
| G protein-coupled receptor 77 | GPR77 |
| membrane-associated ring finger (C3HC4) 1 | MAR_1 |
| jun B proto-oncogene | JUNB |
| G protein-coupled receptor 183 | GPR183 |
| chemokine (C motif) receptor 1 | XCR1 |
| transforming, acidic coiled-coil containing protein 3 | TACC3 |
| zinc finger protein 285A | ZNF285A |
| transforming growth factor, beta-induced, 68 kDa | TGFBI |
| Fc receptor-like 2 | FCRL2 |
| BCL2-related protein A1 | BCL2A1 |
| leukocyte immunoglobulin-like receptor, subfamily B (with TM and ITIM | LILRB1 |
| domains), member 1 | |
| elastin microfibril interfacer 2 | EMILIN2 |
| hydrogen voltage-gated channel 1 | HVCN1 |
| GLI-Kruppel family member HKR1 | HKR1 |
| chromosome 11 open reading frame 75 | C11orf75 |
| spermidine synthase | SRM |
| growth factor receptor-bound protein 2 | GRB2 |
| lymphocyte-specific protein tyrosine kinase | LCK |
| cytochrome b-245, alpha polypeptide | CYBA |
| SHC SH2-domain binding protein 1 | SHCBP1 |
| plasminogen activator, urokinase receptor | PLAUR |
| ABI family, member 3 | ABI3 |
| olfactory receptor, family 2, subfamily C, member 1 | OR2C1 |
| cathepsin B | CTSB |
| cyclin Y-like 1 | CCNYL1 |
| RUN domain containing 2C /// RUN domain containing 2B | RUNDC2C /// |
| | RUNDC2B |
| histone cluster 1, H3j | HIST1H3J |
| nuclear factor of kappa light polypeptide gene enhancer in B-cells | NFKBIE |
| inhibitor, epsilon | |
| golgi autoantigen, golgin subfamily a, 2-like 1 | GOLGA2L1 |
| oral-facial-digital syndrome 1 | OFD1 |
| C-type lectin domain family 4, member E | CLEC4E |
| chromosome 2 open reading frame 14 | C2orf14 |
| zinc finger protein 250 | ZNF250 |
| checkpoint with forkhead and ring finger domains | CHFR |
| interleukin 2 receptor, gamma (severe combined immunodeficiency) | IL2RG |
| nucleolar and spindle associated protein 1 | NUSAP1 |
| granulin | GRN |
| prosaposin | PSAP |
| metadherin | MTDH |
| glutamate receptor, ionotropic, N-methyl D-aspartate-associated protein 1 | GRINA |
| (glutamate binding) | |
| cytochrome P450, family 27, subfamily B, polypeptide 1 | CYP27B1 |
| syntaxin binding protein 4 | STXBP4 |
| Gardner-Rasheed feline sarcoma viral (v-fgr) oncogene homolog | FGR |
| Z-DNA binding protein 1 | ZBP1 |
| procollagen-lysine 1,2-oxoglutarate 5-dioxygenase 1 | PLOD1 |
| zinc finger protein 608 | ZNF608 |
| tubulin, alpha 1c | TUBA1C |
| RAB7, member RAS oncogene family-like 1 | RAB7L1 |
| tumor necrosis factor receptor superfamily, member 1B | TNFRSF1B |
| nuclear receptor subfamily 1, group H, member 3 | NR1H3 |
| sialic acid binding Ig-like lectin 10 | SIGLEC10 |
| aurora kinase B | AURKB |
| CD72 molecule | CD72 |
| chromosome 19 open reading frame 61 | C19orf61 |
| profilin 1 | PFN1 |
| CD209 molecule | CD209 |
| GPRIN family member 3 | GPRIN3 |
| septin 6 | SEP_06 |
| mucolipin 2 | MCOLN2 |
| chromosome X open reading frame 42 | CXorf42 |
| guanylate binding protein 5 | GBP5 |
| integrin, alpha M (complement component 3 receptor 3 subunit) | ITGAM |
| carbohydrate (N-acetylglucosamine-6-O) sulfotransferase 2 | CHST2 |
| ATPase, Na+/K+ transporting, beta 3 polypeptide | ATP1B3 |
| EF-hand calcium binding domain 2 | EFCAB2 |
| CD33 molecule | CD33 |
| actin related protein 2/3 complex, subunit 4, 20 kDa /// tubulin tyrosine | ARPC4 /// TTLL3 |
| ligase-like family, member 3 | |
| tweety homolog 2 (Drosophila) | TTYH2 |
| major histocompatibility complex, class II, DR beta 5 | HLA-DRB5 |
| inositol polyphosphate-5-phosphatase, 145 kDa | INPP5D |
| KIAA1328 | KIAA1328 |
| v-yes-1 Yamaguchi sarcoma viral related oncogene homolog | LYN |
| CD180 molecule | CD180 |
| BMS1 pseudogene 4 | BMS1P4 |
| Rap guanine nucleotide exchange factor (GEF) 1 | RAPGEF1 |
| ubiquitin-conjugating enzyme E2, J1 (UBC6 homolog, yeast) | UBE2J1 |
| Epstein-Barr virus induced 3 | EBI3 |
| phosphoinositide-3-kinase, regulatory subunit 5 | PIK3R5 |
| CD74 molecule, major histocompatibility complex, class II invariant chain | CD74 |
| ring finger protein 19B | RNF19B |
| interleukin 22 receptor, alpha 2 | IL22RA2 |
| major histocompatibility complex, class I, C /// major histocompatibility | HLA-C /// HLA-B |
| complex, class I, B | |
| olfactory receptor, family 51, subfamily I, member 2 | OR51I2 |
| superoxide dismutase 2, mitochondrial | SOD2 |
| sialophorin | SPN |
| CD3g molecule, gamma (CD3-TCR complex) | CD3G |
| SLAM family member 7 | SLAMF7 |
| zinc finger protein 512 | ZNF512 |
| lymphocyte cytosolic protein 2 (SH2 domain containing leukocyte protein | LCP2 |
| of 76 kDa) | |
| adrenergic, beta, receptor kinase 1 | ADRBK1 |
| sorting nexin 10 | SNX10 |
| prostaglandin D2 synthase 21 kDa (brain) | PTGDS |
| SAM and SH3 domain containing 3 | SASH3 |
| chemokine (C-C motif) receptor 7 | CCR7 |
| similar to mCG49427 | LOC100130130 |
| family with sequence similarity 27-like | FAM27L |
| osteoclast associated, immunoglobulin-like receptor | OSCAR |
| nuclear factor of kappa light polypeptide gene enhancer in B-cells 2 | NFKB2 |
| (p49/p100) | |
| inhibitor of growth family, member 5 | ING5 |
| lipoma HMGIC fusion partner-like 2 | LHFPL2 |
| selectin P ligand | SELPLG |

1. A method for determining the prognosis of pulmonary sarcoidosis in an individual subject, comprising conducting gene expression analysis on a sample from the subject, wherein said subject has or is suspected of having pulmonary sarcoidosis and said sample is a biopsy obtained by bronchoscopy under procedural methods not requiring general anesthesia.

2. The method of claim 1, wherein the biopsy is a transbrochial biopsy (TBB).

3. The method of claim 1, wherein determining the prognosis of pulmonary sarcoidosis comprises categorising the sarcoidosis as either progressive or self-limiting.

4. The method of claim 3, wherein said categorising is carried out by a method comprising:
(a) measuring the expression level at least one gene set in the sample taken from the subject;
(b) comparing the measured expression level to the expression level for the same at least one gene set as previously determined in
patients confirmed as having progressive sarcoidosis, and/or
patients confirmed as having self-limiting sarcoidosis and
(c) thereby categorising the sarcoidosis in said subject as either progressive or self-limiting.

5. The method of claim 4, wherein (c) comprises categorising the sarcoidosis as:
progressive; if the expression level of the at least one gene set in the sample from the subject is significantly different to the corresponding expression level in patients confirmed as having self-limiting sarcoidosis, or if the expression level of the at least one gene set is not significantly different to the corresponding expression level in patients confirmed as having progressive sarcoidosis; or
self-limiting; if the expression level of the at least one gene set in the sample from the subject is significantly different to the corresponding expression level in patients confirmed as having progressive sarcoidosis, or if the expression level of the at least one gene set is not significantly different to the corresponding expression level in patients confirmed as having self-limiting sarcoidosis.

6. The method of claim 1, wherein the subject exhibits one or more of the following symptoms:
Symptoms associated with the lung: selected from shortness of breath, wheezing, hoarseness, dry cough with phlegm, chest pain, and tightness in the chest;
Symptoms associated with the eye: selected from uveitis, uveoparotitis, iridocyclitis, retinal inflammation, loss of visual acuity or blindness, eye dryness, redness, tearing, burning or itching, and photophobia;
Cutaneous symptoms: selected from range from rashes, noduli, erythema nodosum and lupus pernio;
General symptoms: selected from joint and muscle pain irregular heartbeat, loss of sensation, loss of muscle strength, headache, dizziness, weight loss, fever and fatigue.

7. The method of claim 1, wherein a chest radiograph of the subject has been assigned any one of stages 1 to 4, wherein:
Stage 1: bilateral hilar lymphadenopathy (BHL), which may be accompanied by paratracheal adenopathy. Lung fields are clear of infiltrates.
Stage 2: bilateral hilar adenopathy (BHL) accompanied by parenchymal infiltration;
Stage 3: parenchymal infiltration without bilateral hilar adenopathy (BHL)
Stage 4: advanced pulmonary fibrosis with evidence of honey-combing, hilar retraction, bullae, cysts, and emphysema.

8. A method of slowing or preventing the development of pulmonary fibrosis in an individual sarcoidosis patient by identifying patients with worse prognosis for further treatment by:
(i) assessing the prognosis of pulmonary sarcoidosis in the subject using a method of claim 1; and
(ii) administering to a subject having sarcoidosis categorised in (i) as progressive, a therapeutically effective amount of an agent suitable for the treatment of pulmonary fibrosis.

9. The method of claim 8 wherein the agent is a corticosteroid, new generation biologic or an immunosuppressive agent.

10. A test kit for use in a method for determining the prognosis of pulmonary sarcoidosis in an individual subject, which test kit comprises one or more agents suitable for analysing gene expression in a sample taken from the subject.

11. The kit of claim 10 wherein the agent is at least one microarray comprising a probe to one or more of the genes in the human genome.

12. A method for determining the prognosis of pulmonary sarcoidosis in an individual subject, comprising conducting gene expression analysis on a sample from the subject, wherein said subject has or is suspected of having pulmonary sarcoidosis and wherein said gene expression analysis comprises determining the expression level of:
the gene set consisting of the genes listed in Table 1 or Table 3; or
at least one gene set from those listed in Table 2 or Table 5; or
at least one gene set selected from the “immune response”, “immune system process”, “lymphocyte activation”, “defence response”, and “leukocyte activation” gene sets, or any combination of said gene sets; or
at least one gene set from each of the major categories shown in Table 5.

13. The method of claim 12, wherein determining the expression level of a gene set comprises measuring the expression level of at least 50% of the genes in said gene set.
